(19) United States
(12) Patent Application Publication
(10) Pub. No.: US 2003/0054419 A1
(43) Pub. Date: Mar. 20, 2003

(54) Method to determine prognosis after therapy for prostate cancer

(76) Inventors: Kevin M. Slawin, Houston, TX (US); Shahrokh Shariat, Houston, TX (US)

Correspondence Address: SCHWEGMAN, LUNDBERG, WOESSNER & KLUTH, P.A., P.O. BOX 2938, MINNEAPOLIS, MN, 55402, US

(21) Appl. No.: 10/071,861

(22) Filed: Feb. 7, 2002

Related U.S. Application Data
(60) Provisional application No. 60/266,976, filed on Feb. 7, 2001.
(60) Provisional application No. 60/295,498, filed on Jun. 1, 2001.
(60) Provisional application No. 60/295,692, filed on Jun. 4, 2001.

(51) Int. Cl. G01N 33/574

(52) U.S. Cl. 435/7.23

(57) ABSTRACT

A method for prognosis of patients with prostate cancer, e.g., clinically localized prostate cancer, is provided.

### CROSS REFERENCE TO RELATED APPLICATIONS

This application claims the benefit of the filing date of U.S. application Ser. No. 60/266,976, filed Feb. 7, 2001, U.S. application Ser. No. 60/295,498, filed Jun. 1, 2001, and U.S. application Ser. No. 60/295,692, filed Jun. 4, 2001, the disclosures of which are incorporated by reference herein.

### STATEMENT OF GOVERNMENT RIGHTS

The invention was made at least in part with a grant from the Government of the United States of America (grant no. CA 58203 from the National Institutes of Health). The Government has certain rights to the invention.

### BACKGROUND OF THE INVENTION

Prostate cancer is the most commonly diagnosed cancer and the second leading cause of cancer death for men in the United States. In 1999, an estimated 179,300 men were diagnosed with prostate cancer and 37,000 died of this disease. Despite the identification of several new potential biomarkers for prostate cancer (e.g., p53, p21, p27, and E-cadherin), prostate specific antigen (PSA) and the histologic Gleason score have remained the most powerful predictors of prostate cancer biology. In fact, the widespread use of PSA-based screening has dramatically increased the number of men diagnosed and treated for clinically localized prostate cancer over the past decade. Concomitantly the incidence of clinical metastatic disease at the time of initial diagnosis has dropped considerably, in concert with an overall decrease in prostate cancer mortality (Merill et al., 2000).

Even given the significant rate of long-term cancer control afforded patients with clinically localized prostate cancer treated with radical prostatectomy or radiation therapy, approximately 30% of these patients will fail treatment, as evidenced by a detectable or rising PSA, which often is due to early dissemination of microscopic metastatic disease prior to primary therapy (Pound et al., 1997). Conventional staging modalities such as bone scan, CT scan, and MRI have a limited role in staging patients with clinically localized prostate cancer, because of their poor performance in detecting early, low-volume metastases (Oesterling et al., 1993; Engeler et al., 1992). Pre-operative nomograms that consider established markers such as PSA, clinical stage, and biopsy Gleason score can provide an estimate of the risk of nodal metastasis or disease recurrence, but are still imperfect for determining the pathological stage or prognosis in individual patients (Partin et al., 1997; Kattan et al., 1998). Improved pre-operative identification of patients with occult metastatic disease, who have a high probability of developing disease progression despite effective local therapy, would be helpful in sparing men from the morbidity of a radical prostatectomy or radiation therapy that would be ineffective or for selecting patients best suited for clinical trials of neoadjuvant or adjuvant therapy.

One example of a molecule which has been investigated for its association with cancer is transforming growth factor β₁ (TGF-β₁), a pleiotropic growth factor that regulates cellular proliferation, chemotaxis, cellular differentiation, immune response, and angiogenesis. Loss of response to the inhibitory effect of TGF-β₁ has been associated with the progression of cancer. For example, increased local expression of TGF-β₁ has been associated with tumor grade, pathological stage, and lymph node metastasis in patients with prostate cancer (Steiner et al., 1992; Eastham et al., 1995; Truong et al., 1993; Thompson et al., 1992). In addition, elevated circulating levels of TGF-β₁ have been found in patients with a variety of different tumors (Wakefield et al, 1995; Kong et al., 1999; Shirai et al., 1994; Eder et al., 1996; Junker et al., 1996). Although higher circulating TGF-β₁ levels have shown an association with prostate cancer invasion (Ivanovic et al., 1995) and metastasis in some studies (Ivanovic et al., 1995; Adler et al., 1999; Kakehi et al, 1996), other studies have not shown such an association (Wolff et al., 1999; Perry et al., 1997). Thus, it is unclear whether circulating TGF-β₁ levels are associated with prostate cancer invasion and metastases.

Insulin-like growth factors (IGFs) are potent mitogens that enhance cell growth and proliferation. Paracrine stimulation of the IGF-I signaling pathway has been implicated in the progression of prostate cancer. IGF binding proteins (IGFBPs) function indirectly by regulating IGF bioavailability, but also have direct IGF-independent effects. Increased circulating levels of IGFBP-2 have been observed in prostate cancer and low IGFBP-3 levels have been associated with increased prostate cancer risk, however, the relative importance of systemic levels of IGFs and IGFBPs in prostate cancer remains unclear.

Interleukin-6 (IL-6) is a molecule that regulates the growth and differentiation of various types of malignant tumors, including prostate carcinomas. Circulating levels of IL-6 have been shown to be elevated in patients with locally advanced and metastatic prostate cancer. IL-6 signaling occurs through a receptor complex consisting of a specific receptor and a signal-transducing component (gp130). The soluble form of the IL-6 receptor (IL-6sR), which arises from proteolytic cleavage of membrane-bound IL-6 receptor, can augment IL-6 induced signaling by facilitating the binding of the IL-6/IL-6sR complex to membrane-bound gp130.

While a number of molecules other than PSA are associated with prostate cancer, it is unclear whether any of these molecules are useful to predict disease outcome. Thus, what is needed is a method to predict disease outcome for patients with clinically localized prostate cancer, e.g., one that is independent of other markers for prostate cancer and/or may be employed with other markers to better define pathological stage.

### SUMMARY OF THE INVENTION

The invention provides a method to predict the disease-free status of a prostate cancer patient after therapy. In one embodiment of the invention, the method comprises contacting a physiological fluid sample from a patient prior to or after therapy for clinically localized prostate cancer with an agent that binds to TGF-β₁ so as to form a complex. Thus, in one embodiment of the invention, the method comprises contacting a physiological fluid sample from a patient after therapy for prostate cancer, e.g., a patient with clinically localized prostate cancer or having a clinical stage≦T3a, with an agent that binds to TGF-β₁ so as to form a complex. When the sample is collected “after” therapy for use in the methods of the invention, it may be collected at times up to five to six months or less, e.g., one, one and a half, two, three or four months, after therapy, including from one, two or more, e.g., three, four or five, days after therapy, up to one week after therapy, or more, e.g., at two or three weeks. The amount or level of complex formation is then correlated to the risk of non-prostate confined disease or disease progression in the patient. In one embodiment, the fluid sample is a blood sample and more preferably a plasma sample. Preferably, the sample is obtained from a patient that has not received any previous therapy for prostate cancer, e.g., hormonal therapy, radiation therapy or brachytherapy. Preferred agents that bind to TGF-β₁ include, but are not limited to, antibodies specific for TGF-β₁ and the TGF-β₁ receptor protein, e.g., type I or II. As used herein, a sample of “physiological body fluid” includes, but is not limited to, a sample of blood, plasma, serum, seminal fluid, urine, saliva, sputum, semen, pleural effusions, bladder washes, bronchioalveolar lavages, cerebrospinal fluid and the like. As used herein, a patient with “clinically localized prostate cancer” means that the patient has no detectable metastases, e.g., detectable by MRI, bone scan, CT scan, or PET scan.

As described herein, the relationship between pre-operative or post-operative platelet-poor plasma TGF-β₁ levels and established markers of prostate cancer invasion, metastasis, and disease progression was determined in a large consecutive cohort of patients with prostate cancer, e.g., those undergoing radical prostatectomy. One study group consisted of 120 consecutive patients who underwent radical prostatectomy (median follow-up of 53.8 months) for clinically localized prostate cancer. Pre-operative platelet-poor plasma levels of TGF-β₁ were measured and correlated with clinical and pathological parameters. TGF-β₁ levels were also measured in 44 healthy men without any cancer, in 19 men with prostate cancer metastatic to regional lymph nodes, and in 10 men with prostate cancer metastatic to bone. None of the patients were treated with hormonal or radiation therapy prior to sample collection.

plasma TGF-β₁ levels in patients with lymph node metastases (14.2±2.6 ng/mL) and bone metastases (15.5±2.4 ng/mL) were significantly higher than those in radical prostatectomy patients (5.2±1.3 ng/mL) and healthy subjects (4.5±1.2 ng/mL) (P values<0.001). Pre-operative plasma TGF-β₁ levels and biopsy Gleason grade were both significant independent predictors of organ-confined disease (P=0.006 and P=0.006, respectively) and PSA progression (P<0.001 and P=0.021, respectively). Within each pathological stage, patients who developed biochemical progression had significantly higher TGF-β₁ levels than those who remained disease-free after surgery (P values<0.001). In patients who progressed, pre-operative plasma TGF-β₁ levels were significantly higher in those with presumed distant versus local-only failure (P=0.019). In men without clinical or pathological evidence of metastases, the pre-operative plasma TGF-β₁ levels were the strongest predictor of biochemical progression after surgery, likely due to an association with occult metastatic disease present at the time of radical prostatectomy.

As also described herein, a larger cohort of 468 radical prostatectomy patients were employed to study marker interactions. Of these patients, 278 patients had samples available at 6 to 8 weeks after post-radical prostatectomy. The clinical stage of these patients was≦T3a (47% cT1, 49% cT2, and 4% cT3a) and they had a median PSA of 8.2 ng/mL (range of 0.2 to 60 ng/mL). The median age for these patients was 63 years (range 40 to 81) and the median follow up for them was about 51 months. Fourteen percent (63/468) had PSA recurrence. Post-operative plasma TGF-β₁ levels were found to be useful as a prognostic marker for prostate cancer progression. Thus, serial measurements TGF-β₁ may be particularly useful to monitor the outcome of therapy, e.g., surgery, radiation, or hormonal therapy or brachytherapy, similarly to serial measurements of PSA. Moreover, in a multivariate Cox proportional hazards model, post-therapy measurements of TGF-β₁ were found to be a stronger predictor than pre-therapy measurements of TGF-β₁. Hence, the invention provides a method to determine the risk of progression of a patient after therapy for prostate cancer. The method comprises contacting a blood plasma sample from a patient after therapy for prostate cancer with an agent that binds to TGF-β₁ so as to form a complex. Then the amount or level of complex formation is correlated with the risk of progression.

Thus, the level of TGF-β₁ in body fluids of humans is prognostically useful, and may optionally be employed in conjunction with other markers for neoplastic disease such as those for prostate cancer, e.g., urinary plasminogen activator (UPA), urinary plasminogen activator receptor (UPAR), plasminogen activator inhibitor 1 (PAI-1), IL-6, IL-6sR, IGFBP-2, IGFBP-3, p53, p21, E-cadherin, and PSA, as well as Gleason scores, e.g., in a nomogram to predict stage and outcome in patients with prostate cancer. In one embodiment, the prognosis is based on a computer derived analysis of data of the amount, level or other value (score) for one or more markers for prostate cancer. Data may be input manually or obtained automatically from an apparatus for measuring the amount or level of one or more markers.

Thus, the invention provides a nomogram that employs standard clinical and pathological measures of prostate cancer, as well as one or more serum/plasma proteins, including but not limited to, TGF-β₁, IL6, IL6sR, IGFBP-2 and IGFBP-3 to predict outcomes in clinical situations for prostate cancer patients including pre-prostatectomy, post-prostatectomy, pre-radiation therapy, post-radiation therapy, recurrence after primary therapy, e.g., rising PSA after surgery or radiation therapy and metastatic disease.

The invention also provides a prognostic method. The method comprises contacting a physiological fluid sample from a patient prior to or after primary therapy for clinically localized prostate cancer with an agent that binds to TGF-β₁ so as to form a complex. Then complex formation is detected or determined and the amount or level of complex formation is employed to predict the patient's final pathological stage and/or biochemical progression, e.g., after therapy or in the absence of therapy. Preferably, the sample is a blood sample, and more preferably, a plasma sample.

As also described herein, the pre-operative or post-operative plasma levels of IL-6 and IL-6sR may be correlated with clinical and pathological parameters. Plasma IL-6 and IL-6sR levels in patients with bone metastases were significantly higher than those in healthy subjects, in prostatectomy patients, or in patients with lymph node metastases (P values<0.001). In a pre-operative model that included IL-6 or IL-6sR in addition to Partin nomogram variables, pre-operative plasma IL-6, IL-6sR, and biopsy Gleason score were independent predictors of organ-confined disease (P values≦0.01) and PSA progression (P values≦0.028). However, in an alternative model that included both IL-6 and IL-6sR, only pre-operative plasma IL-6sR remained an independent predictor of PSA progression (P=0.038). Thus, IL-6 and IL-6sR levels are elevated in men with prostate cancer metastatic to bone. In patients with clinically localized prostate cancer, the pre-operative plasma level of IL-6 and IL-6sR are associated with markers of more aggressive prostate cancer and are predictors of biochemical progression after surgery.

Hence, the invention further provides a method in which a physiological fluid sample from a patient prior to or after primary therapy for clinically localized prostate cancer is contacted with an agent that binds to IL-6 or IL-6sR so as to form a complex. Then the amount or level of complex formation is correlated to the risk of non-prostate confined disease (disease progression), final pathological stage and/or biochemical progression. Thus, the level of IL-6 and/or IL-6sR in body fluids of humans is prognostically useful, and may optionally be employed in conjunction with other markers for neoplastic disease such as those for prostate cancer, e.g., UPA, UPAR, PAI-1, TGF-β₁, IGFBP-2, IGFBP-3, p53, p21, E-cadherin, and PSA, as well as Gleason scores, e.g., in a nomogram to predict stage and outcome in patients with prostate cancer. In one embodiment, the prognosis may be based on a computer derived analysis of data of the amount, level or other value for one or more markers for prostate cancer, and data may be input manually or obtained automatically.

As further described herein, pre-operative or post-operative plasma levels of IGF-I, IGFBP-2, and IGFBP-3 may be measured and correlated with clinical and pathological parameters. In the 120 patients, 44 healthy men without any cancer, 19 men with prostate cancer metastatic to regional lymph nodes, and the 10 men with prostate cancer metastatic to bone mentioned hereinabove, it was found that plasma IGFBP-2 levels in prostatectomy patients and in patients with lymph node metastases or bone metastases were significantly higher than those in healthy subjects (P values≦0.006). Plasma IGBP-3 levels in patients with lymph node metastases and bone metastases were significantly lower than those in prostatectomy patients and healthy subjects (P values≦0.031). Pre-operative plasma IGFBP-2 and biopsy Gleason score were both independent predictors of organ-confined disease (P=0.001 and P=0.005, respectively) and PSA progression (P=0.049 and P=0.035, respectively). When adjusted for IGFBP-2, IGFBP-3 was an independent predictor of PSA progression (P=0.040). Thus, while plasma IGFBP-2 levels are elevated in men with prostate cancer, IGFBP-3 levels are decreased in men with prostate cancer metastatic to regional lymph nodes and bone. In patients with clinically localized prostate cancer, the pre-operative plasma IGFBP-2 level is associated with markers of more aggressive prostate cancer and is a predictor of biochemical progression after surgery.

The invention thus provides a method which comprises contacting a physiological fluid sample from a patient prior to or after primary therapy for clinically localized prostate cancer with an agent that binds to IGFBP-2 and optionally to IGFBP-3, so as to form a complex. Complex formation is then detected or determined, and correlated to the risk of non-prostate confined disease), final pathological stage and/or biochemical progression. Similar to the methods described above, the level of IGFBP-2 and/or IGFBP-3 in body fluids of humans is prognostically useful, and may optionally be employed in conjunction with other markers for neoplastic disease such as those for prostate to predict stage and outcome in patients with prostate cancer, e.g., using a computer derived analysis of data of the amount, level or other value for one or more markers for prostate cancer.

The invention also provides an apparatus, comprising: a data input means, for input of test information comprising the level or amount of at least one protein in a sample obtained from a mammal, wherein the protein is selected from the group consisting of TGF-β₁IGFBP-2, IL-6, IL-6sR and IGFBP-3; a processor, executing a software for analysis of the level or amount of the at least one protein in the sample; wherein the software analyzes the level or amount of the at least one protein in the sample and provides the risk of non-prostate confined disease in the mammal.

### BRIEF DESCRIPTION OF THE FIGURES

FIG. 1. Kaplan-Meier estimates of PSA progression-free probability for the 120 patients with clinically localized prostate cancer treated with radical prostatectomy stratified into groups above or below the median TGF-β₁ level of 4.9 ng/mL.

FIG. 2. Box plot of the distribution analysis for TGF-β₁ levels stratified by progression status at 48 months in healthy men without cancer (n=44), consecutive radical prostatectomy patients according to pathologic stage (OC=Organ confined; ECE=Extracapsular extension; SVI=Seminal vesicle involvement; LN Mets=Lymph node metastases) with at least 48 months of follow-up (n=109), men with prostate cancer metastatic to regional lymph nodes (LN Mets, n=19), and men with prostate cancer metastatic to bone (Bone Mets, n=10). Data are presented as median, interquartile and overall range.

FIG. 3. Kaplan-Meier estimates of PSA progression-free probability for the 120 patients with clinically localized prostate cancer treated with radical prostatectomy stratified into groups above or below the median IGFBP-3 level of 3239.8 ng/mL.

FIG. 4. Kaplan-Meier estimates of PSA progression-free probability for the 120 patients with clinically localized prostate cancer treated with radical prostatectomy stratified into groups above or below the median IGFBP-2 level of 437.4 ng/mL.

FIG. 5. Pre-operative and post-operative values for IGF-I, IGFBP-2 and IGFBP-3.

FIG. 6.(A) Kaplan-Meier estimates of PSA progression-free probability for the 120 patients with clinically localized prostate cancer treated with radical prostatectomy stratified into groups above or below the median IL-6 level of 1.9 ng/mL. (B) Kaplan-Meier estimates of PSA progression-free probability for the 120 patients with clinically localized prostate cancer treated with radical prostatectomy stratified into groups above or below the median IL-6 level of 1.9 pg/mL.

FIG. 7. Kaplan-Meier estimates of PSA progression-free probability for the 120 patients with clinically localized prostate cancer treated with radical prostatectomy stratified into groups above or below the median IL-6sR level of 25.4 ng/L.

FIG. 8. Box plot of the distribution analysis for IL-6 levels stratified by progression status at 48 months in healthy men without cancer (n=44), consecutive radical prostatectomy patients according to pathologic stage with at least 48 months of follow-up (n=109), men with prostate cancer metastatic to regional lymph nodes (n=19), and men with prostate cancer metastatic to bone (n=10). Data are presented as median, interquartile and overall range.

FIG. 9. Box plot of the distribution analysis for IL-6sR levels stratified by progression status at 48 months in healthy men without cancer (n=44), consecutive radical prostatectomy patients according to pathologic stage with at least 48 months of follow-up (n=109), men with prostate cancer metastatic to regional lymph nodes (n=19), and men with prostate cancer metastatic to bone (n=10). Data are presented as median, interquartile and overall range.

FIG. 10. Survival analysis according to the median TGF-β(DMOS=follow-up time since surgery).

FIG. 11. Survival analysis according to the median IL-6sR (DMOS=follow-up time since surgery).

FIG. 12. Multivariate Cox proportional hazards analysis of pre-operative prediction of various factors for PSA-progression free survival in 468 prostatectomy patients.

FIG. 13. Comparison of predictive value of pre-operative versus post-operative levels of various factors for PSA-progression free survival in 468 prostatectomy patients.

### DETAILED DESCRIPTION OF THE INVENTION

The invention in its broadest sense is a method for predicting organ confined (local) disease status or the potential for progression of prostate cancer following primary therapy, e.g., the presence of occult metastases. The method is particularly useful for evaluating patients at risk for recurrence of prostate cancer following primary therapy for prostate cancer. Specifically, the detection of pre- or post-operative TGF-β₁, IL-6, IL-6sR, IGFBP-2 or IGFBP-3 levels alone, or in conjunction with other markers for prostate cancer, may be useful in predicting organ-confined disease status or the potential for progression in patients with clinically localized prostate cancer.

Non-invasive prognostic assays are provided by the invention to detect and/or quantitate TGF-β₁, IL-6, IL-6sR, IGFBP-2 or IGFBP-3 levels in the body fluids of mammals, including humans. Thus, such an assay is useful in prognosis of prostate cancer. Moreover, such assays provide valuable means of monitoring the status of the prostate cancer. In addition to improving prognostication, knowledge of the disease status allows the attending physician to select the most appropriate therapy for the individual patient. For example, patients with a high likelihood of relapse can be treated rigorously. Because of the severe patient distress caused by the more aggressive therapy regimens as well as prostatectomy, it would be desirable to distinguish with a high degree of certainty those patients requiring aggressive therapies as well as those which will benefit from prostatectomy.

The body fluids that are of particular interest as physiological samples in assaying for TGF-β₁, IL-6, IL-6sR, IGFBP-2 or IGFBP-3 according to the methods of this invention include blood, blood serum, semen, saliva, sputum, urine, blood plasma, pleural effusions, bladder washes, bronchioalveolar lavages, and cerebrospinal fluid. Blood, serum and plasma are preferred, and plasma, such as platelet-poor plasma, are the more preferred samples for use in the methods of this invention.

Exemplary means for detecting and/or quantitating TGF-β₁, IL-6, IL-6sR, IGFBP-2 or IGFBP-3 levels in mammalian body fluids include affinity chromatography, Western blot analysis, immunoprecipitation analysis, and immunoassays, including ELISAs (enzyme-linked immunosorbent assays), RIA (radioimmunoassay), competitive EIA or dual antibody sandwich assays. In such immunoassays, the interpretation of the results is based on the assumption that the TGF-β₁, IL-6, IL-6sR, IGFBP-2 or IGFBP-3 binding agent, e.g., a TGF-β₁, IL-6, IL-6sR, IGFBP-2 or IGFBP-3 specific antibody, will not cross-react with other proteins and protein fragments present in the sample that are unrelated to TGF-β₁, IL-6, IL-6sR, IGFBP-2 or IGFBP-3. Preferably, the method used to detect TGF-β₁, IL-6, IL-6sR, IGFBP-2 or IGFBP-3 levels employs at least one TGF-β₁, IL-6, IL-6sR, IGFBP-2 or IGFBP-3 specific binding molecule, e.g., an antibody or at least a portion of the ligand for any of those molecules. Immunoassays are a preferred means to detect TGF-β₁, IL-6, IL-6sR, IGFBP-2 or IGFBP-3. Representative immunoassays involve the use of at least one monoclonal or polyclonal antibody to detect and/or quantitate TGF-β₁, IL-6, IL-6sR, IGFBP-2 or IGFBP-3 in the body fluids of mammals. The antibodies or other binding molecules employed in the assays may be labeled or unlabeled. Unlabeled antibodies may be employed in agglutination; labeled antibodies or other binding molecules may be employed in a wide variety of assays, employing a wide variety of labels.

Suitable detection means include the use of labels such as radionuclides, enzymes, fluorescers, chemiluminescers, enzyme substrates or co-factors, enzyme inhibitors, particles, dyes and the like. Such labeled reagents may be used in a variety of well known assays. See for example, U.S. Pat. Nos. 3,766,162, 3,791,932, 3,817,837, and 4,233,402.

Still further, in, for example, a competitive assay format, labeled TGF-β₁, IL-6, IL-6sR, IGFBP-2 or IGFBP-3 peptides and/or polypeptides can be used to detect and/or quantitate TGF-β₁, IL-6, IL-6sR, IGFBP-2 or IGFBP-3, respectively, in mammalian body fluids. Also, alternatively, as a replacement for the labeled peptides and/or polypeptides in such a representative competitive assay, labeled anti-idiotype antibodies that have been prepared against antibodies reactive with TGF-β₁, IL-6, IL-6sR, IGFBP-2 or IGFBP-3 can be used.

It can be appreciated that certain molecules such as TGF-β₁ may be present in various forms, e.g., latent and active, as well as fragments thereof, and that these various forms may be detected and/or quantitated by the methods of the invention if they contain one or more epitopes recognized by the respective binding agents. For example, in a sandwich assay where two antibodies are used as a capture and a detection antibody, respectively, if both epitopes recognized by those antibodies are present on at least one form of, for example, TGF-β₁, the form would be detected and/or quantitated according to such an immunoassay. Such forms which are detected and/or quantitated according to methods of this invention are indicative of the presence of the active form in the sample.

For example, TGF-β₁, IL-6, IL-6sR, IGFBP-2 or IGFBP-3 levels may be detected by an immunoassay such as a “sandwich” enzyme-linked immunoassay (see Dasch et al., 1990; Danielpour et al., 1989; Danielpour et al., 1990; Lucas et al., 1990; Thompson et al., 1989; and Flanders et al., 1989). A physiological fluid sample is contacted with at least one antibody specific for TGF-β₁, IL-6, IL-6sR, IGFBP-2 or IGFBP-3 to form a complex with said antibody and TGF-β₁, IL-6, IL-6sR, IGFBP-2 or IGFBP-3. Then the amount of TGF-β₁ in the sample is measured by measuring the amount of complex formation. Representative of one type of ELISA test is a format wherein a solid surface, e.g., a microtiter plate, is coated with antibodies to TGF-β₁, IL-6, IL-6sR, IGFBP-2 or IGFBP-3 and a sample of a patient's plasma is added to a well on the microtiter plate. After a period of incubation permitting any antigen to bind to the antibodies, the plate is washed and another set of TGF-β₁, IL-6, IL-6sR, IGFBP-2 or IGFBP-3 antibodies, e.g., antibodies that are linked to a detectable molecule such as an enzyme, is added, incubated to allow a reaction to take place, and the plate is then rewashed. Thereafter, enzyme substrate is added to the microtiter plate and incubated for a period of time to allow the enzyme to catalyze the synthesis of a detectable product, and the product, e.g., the absorbance of the product, is measured.

It is also apparent to one skilled in the art that a combination of antibodies to TGF-β₁, IL-6, IL-6sR, IGFBP-2 or IGFBP-3 can be used to detect and/or quantitate the presence of TGF-β₁, IL-6, IL-6sR, IGFBP-2 or IGFBP-3 in the body fluids of patients. In one such embodiment, a competition immunoassay is used, wherein TGF-β₁, IL-6, IL-6sR, IGFBP-2 or IGFBP-3 is labeled, and a body fluid is added to compete the binding of the labeled TGF-β₁, IL-6, IL-6sR, IGFBP-2 or IGFBP-3 to antibodies specific for TGF-β₁, IL-6, IL-6sR, IGFBP-2 or IGFBP-3. Such an assay could be used to detect and/or quantitate TGF-β₁, IL-6, IL-6sR, IGFBP-2 or IGFBP-3.

Thus, once binding agents having suitable specificity have been prepared or are otherwise available, a wide variety of assay methods are available for determining the formation of specific complexes. Numerous competitive and non-competitive protein binding assays have been described in the scientific and patent literature and a large number of such assays are commercially available. Exemplary immunoassays which are suitable for detecting a serum antigen include those described in U.S. Pat. Nos. 3,791,932; 3,817,837; 3,839,153; 3,850,752; 3,850,578; 3,853,987; 3,867,517; 3,879,262; 3,901,654; 3,935,074; 3,984,533; 3,996,345; 4,034,074; and 4,098,876. Methods to detect TGF-β₁ levels as well as TGF-β₁ binding molecules are well known to the art (see, e.g., U.S. Pat. Nos. 5,216,126, 5,229,495, 5,571,714, and 5,578,703; WO 91/08291; WO 93/09228; WO 93/09800; and WO 96/36349).

The methods of the invention may be employed with other measures of prostate cancer biology to better predict disease-free status or for staging. For example, the following clinical and pathological staging criteria may be used, e.g., clinical and pathological stage, PSA levels, and Gleason scores, although the use of other criteria does not depart from the scope and spirit of the invention.

T0—No evidence of prostatic tumor.

T1—Clinically inapparent tumor, non-palpable nor visible by imaging.

T1a—Tumor is incidental histologic finding with three of fewer microscopic foci. Non-palpable, with 5% or less of TURP chips (trans-urethral resected prostate tissue) positive for cancer.

T1b—Tumor is incidental histologic finding with more than three microscopic foci. Non-palpable, with greater than 5% of TURP chips (trans-urethral resected prostate tissue) positive for cancer.

T1c—Tumor is non-palpable, and is found in one or both lobes by needle biopsy diagnosis.

T2—Tumor is confined within the prostate.

T2a—Tumor present clinically or grossly, limited to the prostate, tumor 1.5 cm or less in greatest dimension, with normal tissue on at least three sides. Palpable, half of 1 lobe or less.

T2b—Tumor present clinically or grossly, limited to the prostate, tumor more than 1.5 cm in greatest dimension, or in only one lobe. Palpable, greater than half of 1 lobe but not both lobes.

T2c—Tumor present clinically or grossly, limited to the prostate, tumor more than 1.5 cm in greatest dimension, and in both lobes. Palpable, involves both lobes.

T3—Tumor extends through the prostatic capsule.

T3a—palpable tumor extends unilaterally into or beyond the prostatic capsule, but with no seminal vesicle or lymph node involvement. Palpable, unilateral capsular penetration.

T3b—palpable tumor extends bilaterally into or beyond the prostatic capsule, but with no seminal vesicle or lymph node involvement. Palpable, bilateral capsular penetration.

T3c—palpable tumor extends unilaterally and/or bilaterally beyond the prostatic capsule, with seminal vesicle and/or lymph node involvement. Palpable, seminal vesicle or lymph node involvement.

T4 —Tumor is fixed or invades adjacent structures other than the seminal vesicles or lymph nodes.

T4a—Tumor invades any of: bladder neck, external sphincter, rectum. T4b—Tumor invades levator muscles and/or is fixed to pelvic wall.

TABLE 1

| Gleason grade in biopsy† | | |
|---|---|---|
| Primary | Secondary | No. patients (%) |
| 1-2 | 1-2 | 108(11.0) |
| 1-2 | 3 | 158(16.1) |
| 3 | 1-2 | 65(6.6) |
| 3 | 3 | 340(34.6) |
| 1-3 | 4-5 | 213(21.7) |
| 4-5 | 1-5 | 99(10.1) |

†Gleason grades 1-2 are well differentiated, 3 is moderately differentiated, 4-5 are poorly differentiated.

TABLE 2

| Pre-operative PSA‡ | No. patients (%) |
|---|---|
| 0.1-4.0 | 217(22.1) |
| 4.1-10.0 | 472(48.0) |
| 10.1-20.0 | 187(19.0) |
| 20.1-100.0 | 107(10.9) |

‡Median serum prostate-specific antigen (PSA) level for all patients. 6.8 ng/mL (range, 0.1-100.0 ng/mL); mean serum PSA level for all patients, 9.9 ng/mL (95% confidence interval = 9.24-10.54 ng/mL).

The invention will be further described by the following non-limiting examples.

### EXAMPLE 1

Materials and Methods

Patient Population

plasma TGF-β₁ levels were assessed in 44 healthy patients without cancer, in 19 men with prostate cancer metastatic to regional lymph nodes, and in 10 patients with bone scan-proven, metastatic prostate cancer. Neither patients with metastatic lymph node disease nor patients with metastatic bone disease were treated with either hormonal or radiation therapy before plasma collection. The healthy non-cancer group was composed of three sets of patients who presented consecutively to the Baylor Prostate Center's weekly prostate cancer screening program. They had no prior history of any cancer or chronic disease, a normal digital rectal examination, and a PSA of less than 2.0 ng/mL, a PSA range that has an estimated probability of prostate cancer detection of less than 1% in the first 4 years after screening (Smith et al., 1996).

One hundred and twenty consecutive patients were also studied who underwent radical prostatectomy for clinically localized prostatic adenocarcinoma (clinical stage T1 to T2) at The Methodist Hospital, Houston, Tex. No patient was treated pre-operatively with either hormonal or radiation therapy, and none had any secondary cancer. The clinical stage was assigned by the operative surgeon according to the 1992 TNM system. The mean patient age in this study was 61.8±7.2 years (median 63.0, range 40 to 76). Serum prostate specific antigen was measured by the Hybritech® Tandem-R assay (Hybritech, Inc., San Diego, Calif.). TGF-β₁ Measurements

Serum and plasma samples were collected on an ambulatory basis at least 4 weeks after transrectal guided needle biopsy of the prostate, typically performed on the morning of the scheduled day of surgery after a typical pre-operative overnight fast. Blood was collected into Vacutainer® CPT™ 8 mL tubes containing 0.1 mL of 1 M sodium citrate anticoagulant (Becton Dickinson Vacutainer Systems, Franklin Lakes, N.J.) and centrifuged at room temperature for 20 minutes at 1500 g. The top layer corresponding to plasma was decanted using sterile transfer pipettes and immediately frozen and stored at −80° C. in polypropylene cryopreservation vials (Nalgene, Nalge Nunc International, Rochester, N.Y.). Prior to assessment, an additional centrifugation step of the plasma at 10,000 g for 10 minutes at room temperature for complete platelet removal was performed. For quantitative measurements of platelet-poor plasma and serum TGF-β₁ levels, a quantitative sandwich enzyme immunoassay (Quantikine® Human TGF-β₁ Elisa kit, R&D Systems, Minneapolis, Minn.) was used, that is specific for TGF-β₁ and does not cross-react with TGF-β₂ or TGF-β₃. Recombinant TGF-β₁ was used as standard. Every sample was run in duplicate, and the mean was used for data analysis. Differences between the two measurements were minimal, as shown the intra-assay precision coefficient of variation of only 4.73±1.87%.

TGF-β₁ Collection Formats

In a preliminary study, TGF-β₁ levels were assessed from three synchronously drawn blood specimens obtained from 10 of the 44 healthy screening patients. Plasma was separated using Vacutainer® K₃ ethylenediaminetetraacetic acid (EDTA) 5 mL tubes containing 0.057 mL of 15% K₃ EDTA solution, and Vacutainer® CPT™ 8 mL tubes containing sodium citrate (Becton Dickinson Vacutainer Systems, Franklin Lakes, N.J.). Serum was separated using Vacutainer® Brand SST Serum Separator® tubes (Becton Dickinson Vacutainer Systems, Franklin Lakes, N.J.). Specimens were centrifuged at room temperature for 20 minutes at 1500 g, and plasma or serum decanted and frozen at −80° C. until assessment. Prior to assay, an additional centrifugation step at 10,000 g for 10 minutes at room temperature was performed. The investigators were blinded to the nature of the collection formats. Analysis of variance was used to determine whether the collection format significantly affected measured TGF-β₁ levels.

Pathological Examination

All prostatectomy specimens were examined pathologically by a single pathologist, who was blinded to clinical outcome. Pelvic lymph nodes were removed in a standard fashion at surgery and examined microscopically for the presence of metastatic prostate cancer. The radical prostatectomy specimens were processed by whole-mount technique, and pathological parameters evaluated as described in Wheeler et al. (1994).

Postoperative Follow-up

Each patient had a digital rectal examination and serum PSA postoperatively every 3 months for the first year, semiannually from the second through the fifth year, and annually thereafter. A staging evaluation, including bone scan, prostascint, or PSA doubling time calculation was performed in 11 of the 15 patients who had PSA progression prior to the administration of salvage radiation or hormonal therapy. Biochemical progression was defined as a sustained elevation, on 2 or more occasions, of PSA>0.2 ng/mL. The date of progression was assigned to the date of the first value>0.2 ng/mL. Two (1.7%) patients had lymph node positive disease at the time of radical prostatectomy, and surgery was consequently aborted prior to prostate removal. These patients were categorized as failures from the day after surgery. Two (1.7%) patients received adjuvant radiation therapy before biochemical progression because of positive surgical margins. One of them subsequently experienced PSA relapse and was categorized as having progression from the date of the first value>0.2 ng/mL. There were 17 failures among the 120 men. PSA relapse was the sole indication of progression in 14 patients, while 3 had clinical, in addition to biochemical evidence of progression. Post-progression serum PSA doubling time was calculated for patients that had biochemical progression and at least three PSA measurements after the date of progression using the formula: DT=log(2)×T/[log(final PSA)-log(initial PSA)], where DT is the serum PSA doubling time, T is the time interval between the initial and final PSA level, final PSA is the pre-radiation PSA level, and initial PSA is the PSA level noted at the time of the post-operative biochemical recurrence. The natural logarithm was used in all logarithmic transformations. Eight (53%) of the patients that progressed were treated with external beam radiation therapy limited to the prostatic fossa at the Methodist Hospital. Radiation was delivered with 15 to 20 MV photons, and the four-fields technique (anteroposterior/posteroanterior and opposing laterals) with customized field sizes was used. Total radiation therapy dose ranged from 60 to 66 Gy, delivered in daily fractions. A complete response to salvage radiation therapy was defined as the achievement and maintenance of an undetectable serum PSA level. Radiation therapy was considered to have failed if the post-radiation serum PSA levels did not fall to, and remain at, an undetectable level.

Statistical Analysis

Analysis of variance was used to assess differences in TGF-β₁ levels. Multiple comparisons were conducted when the overall test was significant (one way ANOVA followed by Fisher's least significant difference). Pre-operative PSA level had a skewed distribution and so was modeled with a log transformation. Clinical stage was evaluated as T1 versus T2 and biopsy Gleason score was evaluated as grade 2 to 6 versus grade 7 to 10. Differences in TGF-β₁ levels between patients who presumably had distant failure and those who presumably had local-only failure were tested by the Mann-Whitney test. Spearman's rank correlation coefficient was used to compare ordinal and continuous variables. Logistic regression was used for multivariate analysis of binary outcome variables. The Kaplan-Meier method was used to calculate survival functions and differences were assessed with the long rank statistic. Multivariate survival analysis was performed with the Cox proportional hazard regression model. Statistical significance in this study was set as P<0.05. All reported P values are two-sided. All analyses were performed with SPSS statistical package (SPSS version 10.0 for Windows).

Results

Impact of Collection Formats on TGF-β₁ Levels

Initially, the effect of the collection format on TGF-β₁ levels was studied. Mean TGF-β₁ levels, measured in Vacutainer®CPT citrate plasma, Vacutainer™K₃ EDTA plasma, and Vacutainer®BrandSST™ serum from synchronously drawn blood specimens of 10 consecutive, healthy screening patients were 4.21±1.16 ng/mL, 8.34±2.94 ng/mL, and 23.89±5.35 ng/mL, respectively (Table 3). TGF-β₁ levels measured in serum were 3-times higher than those in measured in citrate platelet-poor plasma and 6-times higher than those measured in EDTA platelet-poor plasma. Although analysis of variance showed TGF-β₁ inter-collection format differences to be statistically significant (P values<0.001), TGF-β₁ levels measured in specimens collected by all three sample formats were found to be highly correlated with each other (P values<0.001). However, levels of TGF-β₁ measured in specimens from the two platelet-poor plasma formats were the most highly correlated (CC=0.987). Platelet-poor plasma from Vacutainer®CPT™ sodium citrate tubes was used for TGF-β₁ measurements in the study described below.

TABLE 3

| | TGF-β₁ (ng/mL) | |
|---|---|---|
| Collection Format | Mean ± SD* | Range |
| Citrate plasma | 4.21 ± 1.16 | 2.46-5.38 |
| EDTA plasma | 8.34 ± 2.94 | 7.41-16.33 |
| Serum | 23.89 ± 5.35 | 17.28-37.01 |
| Collection Formats | P value† | Correlation Coefficient‡ |
| EDTA plasma and citrate plasma | <0.001 | 0.987 |
| EDTA plasma and serum | <0.001 | 0.789 |
| Citrate plasma and serum | <0.001 | 0.801 |

*SD = Standard Deviation.
†P - values (two-sided) were calculated based on analysis of variance in a randomized complete block design for the assessment of the difference in TGF-β₁ levels between collection formats.
‡Spearman correlation coefficients were used to assess the relationship between different collection formats.

Clinical and Pathological Characteristics

All patients had clinically localized (T1 or T2) disease, and the mean pre-operative TGF-β₁ and PSA levels were 5.4±2.0 ng/mL (median 4.9, range 1.66 to 15.1) and 9.5±6.3 ng/mL (median 8.2, range 2.1 to 49.0), respectively. Nine (7.5%) patients had PSA levels less than 4 ng/mL; 75 (62.5%) had PSA levels greater than or equal to 4 ng/mL and less than 10 ng/mL; and 36 (30.0%) had PSA levels greater than or equal to 10 ng/mL. Clinical and pathological characteristics are listed in Table 4. On univariate analysis, pretreatment TGF-β₁ levels correlated with pre-operative PSA levels (P=0.019) and pathological stage (P<0.001) (Table 5).

TABLE 4

| | |
|---|---|
| Patients | Patients |
| N(%) | N(%) |

Pre-operative Characteristics

| | | | |
|---|---|---|---|
| Clinical stage | | Biopsy Gleason score | |
| cT1 a + b | 1(0.8) | 2-4 | 3(2.5) |
| cT′ c | 41(34.2) | 5-6 | 77(64.2) |
| cT2a | 46(38.3) | 7 | 35(29.2) |
| cT2b | 16(13.3) | 8-10 | 5(4.1) |
| cT2c | 16(13.3) | | |

Postoperative Characteristics

| | | | |
|---|---|---|---|
| Pathological features | | Pathologic | |
| | | Gleason score * | |
| Organ Confined | 79(65.8) | 2-4 | 0(0) |
| ECE only | 33(27.5) | 5-6 | 59(50.0) |
| SVI+ | 8(6.7) | 7 | 56(47.5) |
| LN+ | 2(1.7) | 8-10 | 3(2.5) |
| SM+ | 16(13.3) | | |

ECE = Extracapsular extension.
SVI+ = Seminal vesicle invasion.
LN+ = Lymph node positive.
SM+ = Positive surgical margins.
*Gleason tumor grade unavailable for two patients, who did not undergo a prostatectomy because of grossly positive pelvic lymph nodes at the time of surgery.

TABLE 5

| Parameter | Correlation Coefficient* | P value† |
|---|---|---|
| Age | 0.21 | 0.823 |
| Pre-operative PSA | 0.214 | 0.019 |
| Biopsy Gleason sum | 0.117 | 0.204 |
| Clinical stage | −0.076 | 0.412 |
| Final pathologic stage | 0.344 | <0.001 |
| Final pathologic Gleason sum | 0.087 | 0.348 |

*Spearman's correlation coefficients were used to assess the relationship between TGF-β₁ levels and clinicopathological parameters.
†P-values (two-sided) of the Spearman correlation were determined by Wilcoxon's rank sum.

Final Pathological Stage and Progression as a Function of TGF-β₁ and Other Parameters

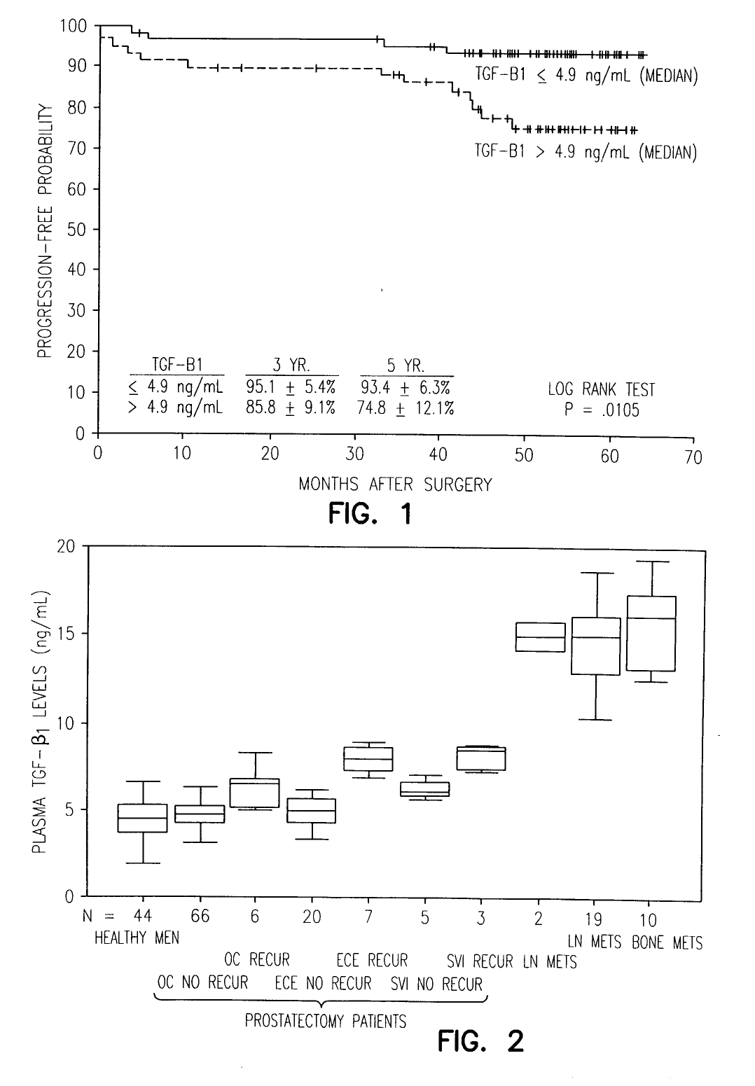

In both an univariate and a multivariate logistic regression analysis that included pre-operative TGF-β₁ pre-operative PSA, clinical stage, and biopsy Gleason score, plasma TGF-β₁ levels (P=0.006; Hazard ratio 0.616, 95% CI 0.436-0.869) and biopsy Gleason grade (P=0.006; Hazard ratio 3.671, 95% CI 1.461-9.219) were significant predictors of organ-confined disease. Overall, only 14% of patients (17 of 120) had cancer progression with a median postoperative follow-up of 53.8 months (range 1.16 to 63.3). The overall PSA progression-free survival was 90.7±5.3% (95% CI) at 3 years and 84.6±6.8% (95% CI) at 5 years. Using the log rank test, it was found that patients with plasma TGF-β₁ levels above the median (4.9 ng/mL) had a significantly increased probability of PSA-progression (P=0.0105; FIG. 1). On univariate Cox proportional hazards regression analysis, plasma TGF-β₁ was associated with the risk of PSA progression (P<0.001) along with biopsy Gleason score (P=0.005, Table 6). In a pre-operative multivariate model that included pre-operative TGF-β₁, pre-operative PSA, clinical stage, and biopsy Gleason score, plasma TGF-β₁ level and Gleason score (P<0.001) were both independent predictors of disease progression.

TABLE 6

| | Univariate | | | Multivariate | | |
|---|---|---|---|---|---|---|
| Variable | Hazard ratio | P | 95% CI | Hazard ratio | P | 95% CI |
| Pre-operative PSA levels* | 5.772 | 0.067 | 0.887-37.547 | 2.408 | 0.363 | 0.362-16.016 |
| Pre-operative TGF-β₁ levels | 2.246 | <0.001 | 1.637-3.083 | 2.268 | <0.001 | 1.629-3.158 |
| Biopsy Gleason Score† | 4.167 | 0.005 | 1.541-11.273 | 3.582 | 0.021 | 1.212-10.585 |
| Clinical Stage‡ | 1.850 | 0.226 | 0.684-5.002 | 1.646 | 0.351 | 0.578-4.687 |

*Preoperative PSA levels were logarithmically transformed.
†Biopsy Gleason Score was categorized as grade 2 to 6 versus grade 7 to 10.
‡Clinical stage was categorized as T1 versus T2.

Characteristics of Patients with Disease Progression

Two of the 17 (12%) patients who progressed had lymph node positive disease at the time of radical prostatectomy. Five patients were presumed to have local failure based on PSA doubling times greater than 12 months (n=3; median 19.6, range 15.8-21.6) or complete response to local salvage radiation therapy (n=2). Eight patients were presumed to have distant failure based on metastatic work-up (positive bone scan or prostascint; n=3), PSA doubling times less than 10 months (n=7; median 6.6, range 1.97-9.80), or failure to respond to local salvage radiation therapy (n=1). Pre-operative plasma TGF-β₁ levels were significantly higher in patients with presumed distant failure (median 8, range 6.5-8.9) than those with local failure (median 5.5, range 4.3-8.3; P=0.019).

TGF-β₁ in Healthy and Metastatic Patients

Mean TGF-β₁ levels in the 44 healthy screening patients, the 19 patients with prostate cancer metastatic to regional lymph nodes, and the 10 patients with metastatic prostate cancer were 4.5±1.2 ng/mL (median 4.70, range 1.0-6.6), 14.24±2.6 ng/mg (median 14.95, range 8.0-19.2), and 15.51±2.4 ng/mL (median 15.20, range 12.4-19.3), respectively. Plasma TGF-β₁ levels in patients with lymph node metastases and bone metastases were significantly higher than those in the initial cohort of 120 prostatectomy patients and healthy subjects (P values<0.001). However, plasma TGF-β₁ levels in the initial cohort of 120 prostatectomy patients were not significantly higher than those in healthy subjects (P=0.053). Similarly, plasma TGF-β₁ levels in patients with bone metastases were not significantly different from those in patients with lymph node metastases (P=0.108).

TGF-β₁ and Prostate Cancer Stage and Progression

FIG. 2 shows box plots of the TGF-β₁ levels in 109 of the 120 consecutive prostatectomy patients who had at least 48 months of follow-up, stratified by progression status at 48 months, 44 healthy men without cancer, 19 men with prostate cancer metastatic to regional lymph nodes, and 10 men with prostate cancer metastatic to bone. TGF-β₁ levels were not different between healthy men, patients with organ confined disease who did not have disease progression, and patients with extracapsular disease who did not have disease progression (P values>0.229). However, TGF-β₁ levels in these three groups were significantly lower than in patients with biochemical progression who had organ confined disease, extracapsular disease, or seminal vesicle invasion, or in patients with lymph node metastases, or patients with bone metastases (P values<0.005). The group of patients with lymph node metastases or bone metastases had similar TGF-β₁ levels (P=0.271), which were significantly higher than those in any of the other groups (P values<0.001).

Discussion

It was confirmed that TGF-β₁ levels are greatly elevated in patients with regional and distant metastases compared to patients with non-metastatic prostate cancer or in healthy subjects. A significant association was found between pre-operative platelet-poor plasma TGF-β₁ levels and established markers of biologically aggressive prostate cancer, such as pre-operative serum PSA levels and final pathologic stage, in a large cohort of consecutive patients with long term follow-up after radical prostatectomy. Furthermore, pre-operative plasma TGF-β₁ was found to be a powerful independent predictor of final pathologic stage and disease progression in patients with clinically localized prostate cancer. Within each pathological stage, patients who developed disease progression had significantly higher TGF-β₁ levels than their non-progressing counterparts. Furthermore, in patients that progressed, pre-operative plasma TGF-β₁ levels were significantly higher in patients with presumed distant failure than those with presumed local-only failure.

In radical prostatectomy patients, the plasma TGF-β₁ level was strongly associated with PSA and pathological stage, two established markers of biologically aggressive prostate cancer. However, in a pre-operative model, TGF-0₁ and biopsy tumor grade but not PSA were independently predictors of advanced pathological stage. An association between elevated TGF-β₁ levels and locally advanced prostate cancer has been previously reported (Ivanovic et al., 1995). In a small pilot study, Ivanovic et al. found that patients with advanced pathological stage had a 2-fold and 4-fold increase in TGF-β₁ levels over patients with confined disease and healthy controls, respectively. However, the majority of patients with organ confined, extracapsular disease, and even seminal vesicle invasion, whose local tumor is completely removed, as evidenced by a negative surgical margin, have long term freedom from biochemical progression (Maru et al., 1999; Epstein et al., 1998; Tefilli et al., 1998; Epstein et al., 2000). On the other hand, most, if not all patients, with lymph node involvement eventually fail local therapy by developing distant metastases, regardless of the success of eradicating local disease (Eastham et al., 2000; Catalona et al., 1998; Walsh et al., 1994). Nomograms consisting of biomarkers that can predict disease progression rather than final pathologic features in patients undergoing radical prostatectomy for prostate cancer would provide greater clinical impact in managing patients with prostate cancer.

A strong association was found between circulating TGF-β₁ levels and disease progression after radical prostatectomy. To process the radical prostatectomy specimens, a whole-mount step-section technique was used that has been shown to be the most accurate means of detecting positive surgical margins and in determining pathologic stage (Wheeler, 1989). In the present study, the positive margin rate was 13.3%, compared with the 16% to 46% positive margin rates reported by others in patients with clinically localized prostate cancer (Ohori et al., 1995; Jones, 1990). Positive surgical margins may suggest the presence of residual local tumor in the surgical bed which has been shown to be a strong predictor of local recurrence (Epstein, et al., 1996). The lower rate of positive surgical margins (13.3%) and the high rate of presumed distant failures (67%) based on PSA doubling times less than 10 months (Pound et al., 1999), the failure to respond to local salvage radiation therapy or a positive metastatic work up, suggested that the association between pre-operative TGF-β₁ levels and disease progression in these patients was more likely to due to an association with the presence of occult metastatic disease present at the time of surgery, rather than with incomplete resection of potentially curable disease. The finding that patients who failed with presumably distant disease had significantly higher TGF-β₁ levels than those who failed locally supports the hypothesis that TGF-β₁ is associated with occult metastases at time of surgery. To further explore this hypothesis, TGF-β₁ levels were analyzed in 109 of the 120 consecutive prostatectomy patients who had at least 48 months of follow-up, stratified by progression status by 48 months and it was found that pre-operative TGF-β₁ levels were significantly elevated in patients with biochemical progression irrespective of the pathologic stage. Thus, TGF-β₁ could be included in pre-operative nomograms for prediction of progression (Kattan et al., 1998).

To further evaluate the association between TGF-β₁ and metastases, TGF-β₁ levels were assessed in ten patients with bone-scan proven metastatic disease, in 19 men with prostate cancer metastatic to regional lymph nodes, and 44 healthy men without any cancer. In agreement with all, except one, previous reports, dramatically elevated levels of TGF-β₁ were found in patients with distant prostate cancer metastases (Ivanovic et al., 1995; Adler et al., 1999; Kakehi et al., 1996). The only study that did not detect any association between TGF-β₁ levels and metastases relied on serum samples, which can lead to aberrant TGF-β₁ levels (Wolff et al., 1999). Furthermore, Wolff et al. (1999) did not specify whether any of the metastatic patients were undergoing androgen-deprivation therapy. The present study evaluated patients with metastatic prostate cancer prior to any therapy, including hormonal therapy. To date, only one other group investigated the levels of TGF-β₁ in patients with regional nodal metastases. In agreement with the present findings, Kakehi et al. (1996) detected significantly elevated TGF-β₁ levels in patients with prostate cancer metastatic to regional lymph nodes. However, in contrast to previous studies (Ivanovic et al., 1995; Adler et al., 1999; Kakehi et al., 1996), no overlap was found between TGF-β₁ levels of regional or distant metastatic patients and those from controls or patients with either localized or advanced prostate cancer. The complete separation of TGF-β₁ levels between patients with clinical or pathological evidence of metastatic disease supports the potential use of plasma TGF-β₁ as a staging marker for prostate cancer that could provide clinically meaningful pathological stratification of the patients. Conversely, in concordance with previous studies, no statistically significant difference was found in plasma TGF-β₁ levels between patients with pathologically localized prostate cancer and healthy men without cancer, limiting the value of TGF-β₁ as a diagnostic tool for early detection of localized prostate cancer (Kakehi et al., 1996; Wolff et al., 1999; Perry et al., 1997).

TGF-β₁ levels were found to be 3 to 6-times higher when measured in serum as compared to platelet-poor plasma. Since TGF-β₁ is present in platelet granules and is released upon platelet activation, the highly elevated levels of TGF-β₁ in serum are likely to derive from damaged platelets and not from the prostate, making quantification of TGF-β₁ in serum erroneous for evaluation of TGF-β₁ originated from or induced by the prostate. To ensure complete platelet removal, an additional centrifugation was performed in the present study, as recommended by Adler et al. (1999), and almost identical amounts of plasma TGF-β₁ were observed. While, as expected, TGF-β₁ values in the serum format were only weakly correlated with those in the plasma formats (correlation coefficients, 0.79 and 0.80), the plasma formats were strongly correlated with each other (correlation coefficient, 0.99). The 2-times lower TGF-β₁ values obtained with the citrate plasma as compared to the EDTA plasma collection format may be due to dilution of the top plasma layer primarily by 1.0 mL of 0.1 mol/L sodium citrate anticoagulant, in the Vacutainer®CPT™ tubes.

This study was limited by the low rate of disease progression in the patient cohort (14%) after a median follow-up of 53.8 months, yielding an estimated 5 year progression-free probability of 85%. The low progression rate in the above-described population may be due to the lower cancer stage and volume observed in more recent surgical series that has accompanied the increasing awareness of prostate cancer in the general population and the wide availability of PSA based screening (Farkas et al., 1998). In other reported series, approximately 44% to 47% of men undergoing radical prostatectomy had pathologically non-organ-confined disease (Partin et al., 1993; Wheeler et al., 1998), while in the present cohort, only 34.2% of cancers were not organ-confined. The pathologic stage of prostate cancer is known to be a strong predictor of progression after radical prostatectomy (Epstein et al., 1996). Nevertheless, 92.5% of the present patients had a pre-operative PSA level above 4 ng/mL; 32.5% had extraprostatic extension in their pathologic prostatectomy specimen, and 50% had a final pathological Gleason score of 7 and above, representative of patients undergoing radical prostatectomy for clinically localized prostate cancer. In addition to a slightly more favorable profile in pathological parameters in the above-described study cohort, the lower progression rate may be due to differences in surgical technique (Ohori et al., 1995; Epstein et al., 1996). The positive margin rate in the present series was 13.3% compared with the 16% to 46% positive margin rates reported by others in patients with clinically localized prostate cancer (Ohori et al., 1995; Jones, 1990), which may have decreased the rate of progression due to local failure.

In conclusion, plasma TGF-β₁ levels are markedly elevated in men with prostate cancer metastatic to regional lymph nodes and bone. In men without clinical or pathological evidence of metastases, the pre-operative plasma TGF-β₁ level is the strongest predictor of biochemical progression after surgery likely due to an association with occult metastatic disease present at the time of radical prostatectomy.

### EXAMPLE 2

Materials and Methods

Patient Population

Plasma IGF-I, IGFBP-2, and IGFBP-3 levels were assessed in 44 healthy patients without cancer, in 19 men with prostate cancer metastatic to regional lymph nodes, and in 10 patients with bone scan-proven, metastatic prostate cancer. Neither patients with metastatic lymph node disease nor patients with metastatic bone disease were treated with either hormonal or radiation therapy before plasma collection. The healthy non-cancer group was composed of three sets of consecutive patients who participated in a weekly prostate cancer screening program. They had no prior history of any cancer or chronic disease, a normal digital rectal examination, and a PSA of less than 2.0 ng/mL, a PSA range that has an estimated probability of prostate cancer detection of less than 1% in the first 4 years after screening (Smith, 1996).

Also, 120 consecutive patients were studied who underwent radical prostatectomy for clinically localized prostatic adenocarcinoma (clinical stage T1 to T2) and who had available plasma samples. No patient was treated pre-operatively with either hormonal or radiation therapy, and none had any secondary cancer. The clinical stage was assigned by the operative surgeon according to the 1992 TNM system. The mean patient age in this study was 61.8 i 7.2 years (median 63.0, range 40 to 76). Serum prostate specific antigen was measured by the Hybritech Tandem-R assay (Hybritech, Inc., San Diego, Calif.).

IGF-I, IGFBP-2, and IGFBP-3 Measurements

Serum and plasma samples were collected on an ambulatory basis at least 4 weeks after transrectal guided needle biopsy of the prostate, typically performed on the morning of the scheduled day of surgery after a typical pre-operative overnight fast. Blood was collected into Vacutainer®CPT™ 8 mL tubes containing 0.1 mL of 1 M sodium citrate anticoagulant (Becton Dickinson Vacutainer Systems, Franklin Lakes, N.J.) and centrifuged at room temperature for 20 minutes at 1500 g. The top layer corresponding to plasma was decanted using sterile transfer pipettes and immediately frozen and stored at −80° C. in polypropylene cryopreservation vials (Nalge Nunc, Rochester, N.Y.). For quantitative measurements of serum and plasma IGF-I and IGFBP-3 levels, the DSL-10-5600ACTIVE®IGF-I Elisa kit and the DSL-10-6600ACTIVE®IGFBP-3 Elisa kit were used, respectively (DSL, Webster, Tex.). For quantitative measurements of serum and plasma IGFBP-2 levels, the DSL-7100 IGFBP-2 Radioimmunoassay kit (DSL) was used. Every sample was run in duplicate, and the mean was used for data analysis. Differences between the two measurements were minimal, as shown the intra-assay precision coefficient of variation of only 4.73±1.87% for IGF-I, 6.95±3.86% for IGFBP-2, and 8.78±4.07 for IGFBP-3.

IGFBP-2 and IGFBP-3 Collection Formats

In a preliminary study, IGFBP-2 and IGFBP-3 levels were assessed in three synchronously drawn blood specimens obtained from 10 of the 44 healthy screening patients. Plasma was separated using Vacutainer® K₃ ethylenediaminetetraacetic acid (EDTA) 5 mL tubes containing 0.057 mL of 15% K₃ EDTA solution, and Vacutainer®CPT™ 8 mL tubes containing sodium citrate (Becton Dickinson Vacutainer Systems, Franklin Lakes, N.J.). Serum was separated using Vacutainer Brand SST Serum Separators™ tubes (Becton Dickinson Vacutainer Systems, Franklin Lakes, N.J.). Specimens were centrifuged at room temperature for 20 minutes at 1500 g, and plasma or serum decanted and frozen at −80° C. until assessment. The investigators were blinded to the nature of the collection formats. Analysis of variance was used to determine whether the collection format significantly affected measured IGFBP-2 and IGFBP-3 levels.

Pathological Examination

All prostatectomy specimens were examined pathologically by a single pathologist who was blinded to clinical outcome. Pelvic lymph nodes were removed in a standard fashion at surgery and examined microscopically for the presence of metastatic prostate cancer. The radical prostatectomy specimens were processed by whole-mount technique, and pathological parameters evaluated as previously described (Wheeler, 1994).

Postoperative Follow-up

Each patient was scheduled to have a digital rectal examination and serum PSA postoperatively every 3 months for the first year, semiannually from the second through the fifth year, and annually thereafter. A staging evaluation, including bone scan, prostascint, and/or PSA doubling time calculation was performed in 11 of the 15 patients who had PSA progression prior to the administration of salvage radiation or hormonal therapy. Biochemical progression was defined as a sustained elevation, on 2 or more occasions, of PSA>0.2 ng/mL. The date of progression was assigned to the date of the first value>0.2 ng/mL. Two (1.7%) patients had lymph node positive disease at the time of radical prostatectomy, and surgery was consequently aborted prior to prostate removal. These patients were categorized as failures from the day after surgery. Two (1.7%) patients received adjuvant radiation therapy before biochemical progression because of positive surgical margins. One of them subsequently experienced PSA relapse and was categorized as having progression from the date of the first value>0.2 ng/mL, while the second was censored on the date of the last follow-up examination. There were 17 failures among the 120 men. PSA relapse was the sole indication of progression in 14 patients, while 3 had clinical, as well as biochemical evidence of progression.

Statistical Analysis

Differences in plasma IGFBP-2 and IGFBP-3 levels were assessed using analysis of variance (ANOVA). Multiple comparisons were conducted, when the overall test was significant (one-way ANOVA followed by Fisher's least significant difference). Spearman's rank correlation coefficient was used to compare ordinal and continuous variables. Logistic regression was used for multivariate analysis of binary outcome variables. The Kaplan-Meier method was used to calculate survival functions, and differences were assessed with the long rank statistic. Multivariate survival analysis was performed with the Cox proportional hazard regression model. Pre-operative PSA level had a skewed distribution and therefore was modeled with a log transformation. Clinical stage was evaluated as T1 versus T2 and biopsy Gleason score was evaluated as grade 2 to 6 versus grade 7 to 10. Statistical significance in this study was set as P<0.05. All reported P values are two-sided. All analyses were performed with SPSS statistical package (SPSS version 10.0 for Windows).

Results

Impact of Collection Formats on IGFBP-2 and IGFBP-3 Levels

Initially, the effect of the collection format on IGFBP-2 and IGFBP-3 levels was studied. Mean IGFBP-2 and IGFBP-3 levels, measured in Vacutainer®CPT™ citrate plasma, Vacutainer®K₃ EDTA plasma, and Vacutainer®BrandSST™ serum from synchronously drawn blood specimens of 10 consecutive, healthy screening patients are shown in Table 7. IGFBP-2 and IGFBP-3 levels measured in citrate plasma were 26% and 28%, respectively, lower than those measured in EDTA plasma, and 37% and 39%, respectively, lower than those measured in serum. Although analysis of variance showed IGFBP-2 and IGFBP-3 inter-collection format differences to be statistically significant (P values<0.001), IGFBP-2 and IGFBP-3 levels measured in specimens collected by all three sample formats were found to be highly correlated with each other (P values<0.001). Similarly to previous results on IGF-I (Shariat, 2000), while statistically significant differences were found in absolute IGFBP-2 and IGFBP-3 levels measured in different collection formats, all three collection formats were highly correlated with each other. Plasma from Vacutainer®CPT™ sodium citrate tubes was used for IGF-I, IGFBP-2, and IGFBP-3 measurements in the following study.

TABLE 7

| | IGFBP-2 (ng/mL) | | IGFBP-3 (ng/mL) | |
|---|---|---|---|---|
| Collection Format | Mean | SD* | Mean | SD* |
| Citrate plasma | 359.3 | 18.1 | 3273 | 256 |
| EDTA plasma | 487.9 | 28.4 | 4566 | 376 |
| Serum | 567.8 | 31.0 | 5401 | 430 |
| | P | Correlation | P | Correlation |
| Collection Formats | value† | Coefficient‡ | value† | Coefficient‡ |
| EDTA plasma and citrate | <0.001 | 0.79 | <0.001 | 0.81 |
| plasma | | | | |
| EDTA plasma and serum | <0.001 | 0.70 | <0.001 | 0.72 |
| Citrate plasma and serum | <0.001 | 0.73 | <0.001 | 0.78 |

*SD = Standard Deviation.
†P-values (two-sided) were calculated based on analysis of variance in a randomized complete block design for the assessment of the difference in IGFBP-2 and IGFBP-3 levels between collection formats.
‡Spearman correlation coefficients were used to assess the relationship between different collection formats.

Clinical and Pathological Characteristics

All patients had clinically localized (T1 or T2) disease, and the mean pre-operative TGF-β₁ and PSA levels were 5.4±2.0 ng/mL (median 4.9, range 1.66 to 15.1) and 9.5±6.3 ng/mL (median 8.2, range 2.1 to 49.0), respectively. Nine (7.5%) patients had PSA levels less than 4 ng/mL; 75 (62.5%) had PSA levels greater than or equal to 4 ng/mL and less than 10 ng/mL; and 36 (30.0%) had PSA levels greater than or equal to 10 ng/mL. Clinical and pathological characteristics are listed in Table 8. On univariate analysis (Table 9), pretreatment IGFBP-2 levels correlated with pathological stage (P<0.001) and grade (P=0.025) and IGFBP-3 10 levels correlated with IGF-1 levels (P<0.001).

TABLE 8

| | |
|---|---|
| Patients | Patients |
| N(%) | N(%) |

Pre-operative Characteristics

| | | | |
|---|---|---|---|
| Clinical stage | | Biopsy Gleason score | |
| cT1 a + b | 1(0.8) | 2-4 | 3(2.5) |
| cT1 c | 41(34.2) | 5-6 | 77(64.2) |
| cT2a | 46(38.3) | 7 | 35(29.2) |
| cT2b | 16(13.3) | 8-10 | 5(4.1) |
| cT2c | 16(13.3) | | |

Postoperative Characteristics

| | | | |
|---|---|---|---|
| Pathological | | Pathologic Gleason | |
| features | | score* | |
| Organ Confined | 79(65.8) | 2-4 | 0(0) |
| ECE only | 33(27.5) | 5-6 | 59(50.0) |
| SVI+ | 8(6.7) | 7 | 56(47.5) |
| LN+ | 2(1.7) | 8-10 | 3(2.5) |
| SM+ | 16(13.3) | | |

ECE = Extracapsular extension.
SVI+ = Seminal vesicle invasion.
LN+ = Lymph node positive.
SM+ = Positive surgical margins.
*Gleason tumor grade unavailable for two patients, who did not undergo a prostatectomy because of grossly positive pelvic lymph nodes at the time of surgery.

TABLE 9

| | Pre-operative IGFBP-2 | | Pre-operative IGFBP-3 | |
|---|---|---|---|---|
| | Correlation | | Correlation | |
| Parameter | Coefficient* | P value | Coefficient* | P value |
| Age | 0.092 | 0.316 | −0.066 | 0.472 |
| Pre-operative PSA | 0.064 | 0.490 | −0.131 | 0.153 |
| Pre-operative IGF-I | 0.013 | 0.884 | 0.61 | <0.001 |
| Clinical stage | −0.009 | 0.921 | 0.056 | 0.546 |
| Biopsy Gleason | −0.204 | 0.025 | −0.071 | 0.438 |
| sum | | | | |
| Final pathologic | −0.375 | <0.001 | −0.104 | 0.261 |
| stage | | | | |
| Final pathologic | −0.204 | 0.027 | −0.013 | 0.891 |
| Gleason sum | | | | |

*Spearman's correlation coefficients were used to assess the relationship of IGFBP-2 and IGFBP-3 levels with IGF-I levels and clinico-pathological parameters.

Final Pathological Stage and Progression as a Function of IGFBP-2 and IGFBP-3 and Other Parameters

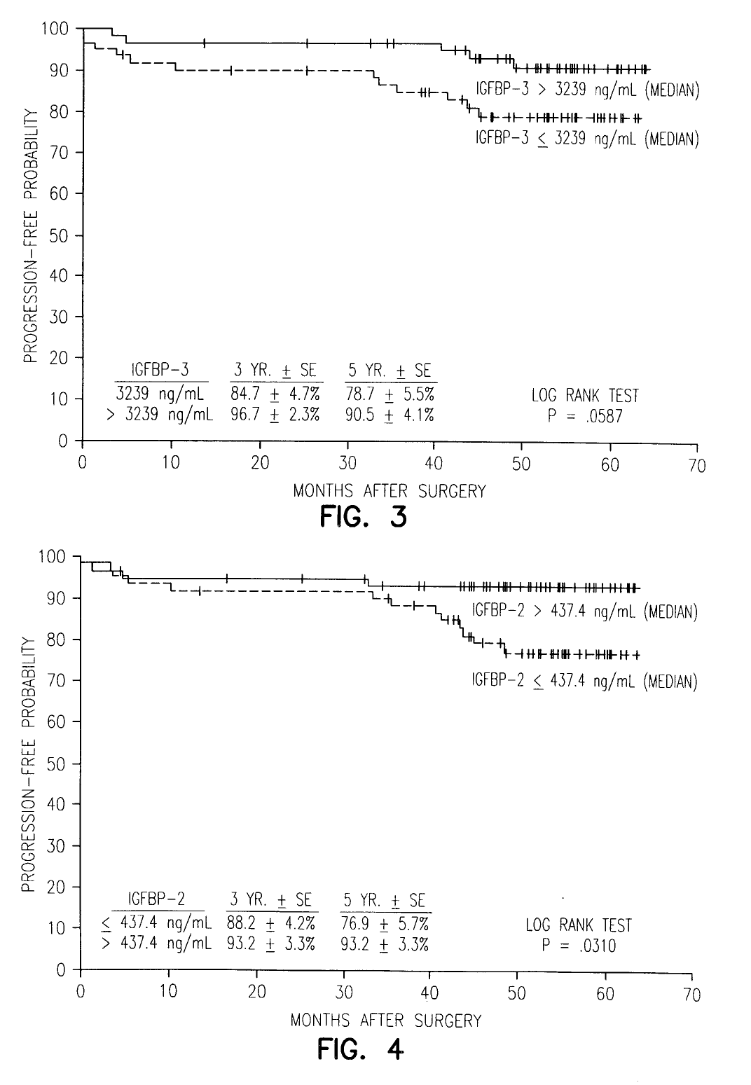

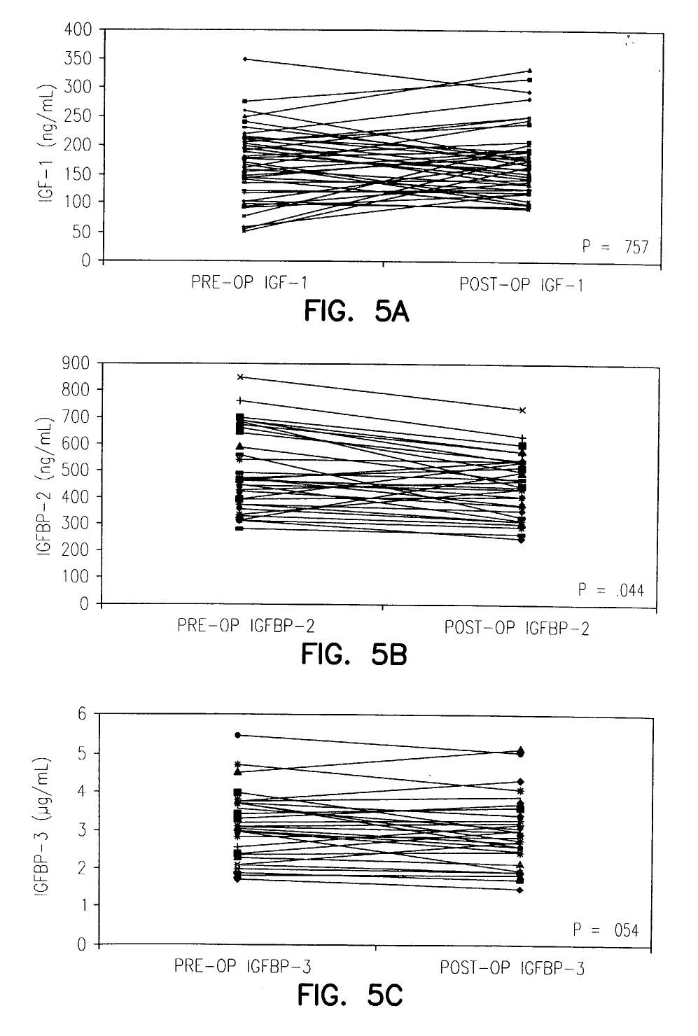

In a multivariate logistic regression analysis, pre-operative plasma IGFBP-2 levels (P=0.001), pre-operative serum PSA levels (P=0.034), and biopsy Gleason grade (P=0.005) were significant predictors of organ-confined disease. Overall, only 14% of patients (17 of 120) had cancer progression with a median postoperative follow-up of 53.8 months (range 1.16 to 63.3). The overall PSA progression-free survival was 90.7±5.3% (95% CI) at 3 years and 84.6±6.8% (95% CI) at 5 years. Using the log rank test, it was found that patients with pre-operative plasma IGFBP-2 levels below the median (437.4 ng/mL) had a significantly increased probability of PSA-progression (P=0.0310; FIG. 3). However, there was no significant difference in PSA-progression-free survival (FIG. 4) between patients stratified by the median level of IGFBP-3 (3239 ng/mL; P=0.0587). On univariate Cox proportional hazards regression analysis (Table 10), plasma IGFBP-2 was associated with the risk of PSA progression (P=0.015) along with biopsy Gleason score (P=0.005). In a pre-operative multivariate model that included pre-operative IGFBP-2, pre-operative PSA, clinical stage, and biopsy Gleason score, plasma IGFBP-2 level and biopsy Gleason score were both independent predictors of disease progression (P=0.049 and P=0.035, respectively). In alternative models where IGBP-2 was replaced by IGF-I, IGFBP-3, or both, biopsy Gleason score was the sole independent predictor of PSA progression (P values<0.09). However when IGFBP-3 level was adjusted for IGFBP-2 level, IGFBP-3 became an independent predictor of disease progression (P values<0.040) and the association of IGFBP-2 with the risk of prostate progression strengthened (P values<0.039). When all three, IGF-I, IGFBP-2, and IGFBP-3 were adjusted for each other, IGFBP-2, IGFBP-3, and biopsy Gleason score were independent predictors of disease progression (P=0.031, P=0.035, and P=0.036, respectively; Table 10).

TABLE 10

| | Univariate | | | Multivariate | | |
|---|---|---|---|---|---|---|
| | Hazard | | | Hazard | | |
| Variable | ratio | P | 95% CI | ratio | P | 95% CI |
| Pre-operative IGF-I | 0.997 | 0.490 | 0.990-1.005 | 1.003 | 0.454 | 0.995-1.012 |
| levels | | | | | | |
| Pre-operative | 0.993 | 0.015 | 0.988-0.999 | 0.994 | 0.031 | 0.988-0.999 |
| IGFBP-2 levels | | | | | | |
| Pre-operative | 0.946 | 0.53 | 0.895-1.001 | 0.926 | 0.035 | 0.836-0.995 |
| IGFBP-3 levels | | | | | | |
| Pre-operative PSA | 5.772 | 0.067 | 0.887-37.547 | 3.671 | 0.124 | 0.699-19.270 |
| levels* | | | | | | |
| Biopsy Gleason | 4.167 | 0.005 | 1.541-11.273 | 3.055 | 0.036 | 1.079-8.654 |
| Score† | | | | | | |
| Clinical Stage‡ | 1.850 | 0.226 | 0.684-5.002 | 1.769 | 0.293 | 0.611-5.122 |

*Pre-operative PSA levels were logarithmically transformed.
†Biopsy Gleason Score was categorized as grade 2 to 6 versus grade 7 to 10.
‡Clinical stage was categorized as T1 versus T2.

Characteristics of Patients with Disease Progression

Of the 17 radical prostatectomy patients who progressed, two (12%) patients had lymph node positive disease at the time of radical prostatectomy. Five patients were presumed to have local failure because their PSA doubling times were greater than 12 months (n=3; median 19.6, range 15.8-21.6) or because they achieved a complete response to local salvage radiation therapy (n=2). Eight patients were presumed to have distant failure because of the results of a metastatic work-up (positive bone scan or prostascint; n=3), because their PSA doubling times were less than 10 months (n=7; median 6.6, range 1.97-9.80), or because they failed to respond to local radiation therapy (n=4). Pre-operative plasma IGF-I levels, IGFBP-2 levels, and IGFBP-3 levels were not significantly different in patients with presumed distant failure than those with local failure (P=0.898, P=0.600, and P=0.059, respectively).

IGFBP-2 and IGFBP-3 in Healthy and Metastatic Patients

Plasma IGF-I levels in 19 patients with prostate cancer metastatic to regional lymph nodes (median 156 ng/mL, range 100-281), in the 10 patients with prostate cancer metastatic to bones (153 ng/mL, range 29-360), in the cohort of 120 prostatectomy patients (median 151 ng/mL, range 42-451), and in the 44 healthy screening patients (median 171 ng/mL, range 62-346) were not significantly different from each other (P=0.413). However, plasma IGFBP-2 levels in the prostatectomy patients (median 437 ng/mL, range 209-871), in the patients with lymph node metastases (median 437 ng/mL, range 299-532), and in the patients with bone metastases (median 407 ng/mL, range 241-592) were significantly higher then those in the healthy subjects (median 340 ng/mL, range 237-495; P values<0.006). Plasma IGFBP-2 levels in patients with clinically localized prostate cancer, with lymph node metastases, or with bone metastases were not significantly different from each other (P values>0.413). Plasma IGFBP-3 levels in patients with lymph node metastases (median 2689 ng/mL, range 1613-3655) and bone metastases (median 2555 ng/mL, range 1549-3213) were significantly lower than those in the cohort of 120 prostatectomy patients (median 3217 ng/mL, range 1244-5452) and in healthy subjects (median 3344 ng/mL, range 1761-5020; P values<0.031). However, plasma IGFBP-3 levels in the prostatectomy patients were not significantly different than those in healthy subjects (P=0.575).

Discussion

IGFBP-2 levels were elevated in patients with non-metastatic and metastatic prostate cancer compared to levels in healthy subjects. A significant association was found between pre-operative plasma IGFBP-2 levels and established markers of biologically aggressive prostate cancer, such as final pathologic stage and grade in patients with clinically localized prostate cancer. Furthermore, pre-operative plasma IGFBP-2 was a robust independent predictor of final pathologic stage and disease progression in a large cohort of consecutive patients with long term follow-up after radical prostatectomy. However, in patients that progressed, pre-operative plasma IGFBP-2 levels were not significantly different in patients with presumed distant failure than those with presumed local-only failure. Plasma IGFBP-3 levels were significantly lower in patients with prostate cancer metastatic to regional lymph nodes and to bones compared to levels in patients with non-metastatic prostate cancer and healthy subjects. While no significant association was found between pre-operative plasma IGFBP-3 levels and established markers of biologically aggressive prostate cancer or disease progression, when adjusted for IGFBP-2 levels, plasma IGFBP-3 was independently associated with prostate cancer progression.

Circulating IGFBP-2 levels are not correlated to circulating IGF-I levels, since more than 90% circulating IGF-I molecules are complexed with IGFBP-3 and a glycoprotein named acid-labile subunit. Most of the circulating IGF-I and IGFBP-3 are produced by the liver and growth hormone stimulates both IGF-I and IGFBP-3 production (Jones, 1995). This growth hormone regulated hepatic release of both IGF-I and IGFBP-3 may explain in part the highly significant but moderate correlation (r=0.61) that was found. Other studies have found an almost identical correlation coefficient.

PSA is an IGFBP-3 protease, capable of acting as a co-mitogen with IGFs in the presence of IGFBP-3 (Cohen, 1992). IGFBP-3 proteolysis by PSA (Cohen, 1994) and cathepsin D (Nunn et al., (1997) likely signify local effects rather then systemic effects, within the prostate or metastatic foci leading to local progression or metastasis growth. Elevated serum PSA level has been correlated with decreased IGFBP-3 (Kanety, 1993).

IGF-I and BPH increase in follow-up doubling the number of cancer-free controls, as well as measurements of IGF-I levels in patients with regional lymph node metastases. Previously, no association was found between circulating IGF-I levels and established markers of biologically aggressive prostate cancer, disease progression, or metastasis. Various independent studies have found no difference in IGF-I levels between patients with prostate cancer and healthy men. Furthermore, a recent study investigating IGF-I levels in a PSA-based screening positive population found IGF-I not to be a useful marker for prostate cancer screening and concluded that high circulating IGF-I level is more likely related to BPH and prostatic enlargement (Finne, 2000), but may be related to prostate cancer risk (early, subclinical disease), but not to cancer biology and prognosis, which more likely results in the disruption of the cellular physiology of IGFs or other growth factors.

While prostate cancer incidence is not increased in patients with acromegaly, the incidence of BPH or enlarged prostate is (Coalo, (1998). Patients with elevated growth hormone who were successfully treated had normal prostate volume and growth hormone deficient subjects had reduced prostate volume. Moreover, IGF-I has been shown to stimulate the growth of BPH derived stromal cells in vitro (Sutkowski et al., (1999).

The mean IGFBP-2 and IGFBP-3 levels measured in Vacutainer®CPT™ citrate plasma were 26% and 28%, respectively, lower than those measured in Vacutainer®K3EDTA plasma, and 37% and 39%, respectively, lower than those measured in Vacutainer®BrandSST serum. The consistent in relative differences measured between the three collection formats for each assay, and the resemblance to relative difference of 27% and 42% for IGFF-I found previously (Shariat, 2000), support that the measurement technique employed was consistent and that the levels of the relative changes of the three markers can be compared. Furthermore this supports that the lower IGF-I, IGFBP-2, and IGFBP-3 values obtained with the Vacutainer®CPT™ citrate plasma as compared to the Vacutainer®K₃EDTA plasma collection format are due to dilution of the top plasma layer primarily by 1.0 mL of 0.1 M sodium citrate anticoagulant. However, although there were statistically significant differences in absolute IGF-I, IGFBP-2, and IGFBP-3 levels measured in serum and in plasma using different collection formats, all three are highly correlated with each other and therefore equally valid as long as the same collection format is used throughout the study.

The complex nature of the IGF axis may require simultaneous measurement of multiple factors in order to fully appreciate the biologic activity of this system. Measurement of other IGFBPs may add to the biological relevance of IGFs in prostate cancer. Other IGFBPs, such as IGFBP-4 and IGFBP-5 have been associated with tumor grade in prostate specimens, and with tumor stage and serum PSA levels in patients. Equally important, IGF-I receptor mediates most of the mitogenic effects of IGFs, and experimental inhibition of the IGF-I receptor has resulted in suppression of adhesion, invasion, and metastases in prostate cancer (Kaplan, 1999). Recent studies suggest that circulating levels of IGFs may not be determinants of tissue bioactivity but rather may vary in parallel with autocrine or paracrine expression within tissues (Yakar, 1999). Since hepatic IGF-I and IGFBP-3 are the major contributors of circulating levels of these two IGFs, important autocrine and paracrine production occurring in other tissues such as the prostate may not be reflected by changes in systemic levels of these molecules.

In conclusion, plasma IGFBP-2 levels are markedly elevated in men with prostate cancer. In men without clinical or pathological evidence of metastases, the pre-operative plasma IGFBP-2 level is a robust predictor of final pathologic stage and biochemical progression after surgery. This association seems, however, not to be due to an association with occult metastatic disease present at the time of radical prostatectomy. On the contrary, pre-operative circulating IGFBP-3 and IGF-1 levels are not independently associated with established markers of biologically aggressive prostate cancer or PSA progression-free survival. The lack of any association with markers of more aggressive prostate cancer or with prostate cancer progression may limit the clinical utility of IGF-I and IGFBP-3 as tumor markers for prostate cancer.

### EXAMPLE 3

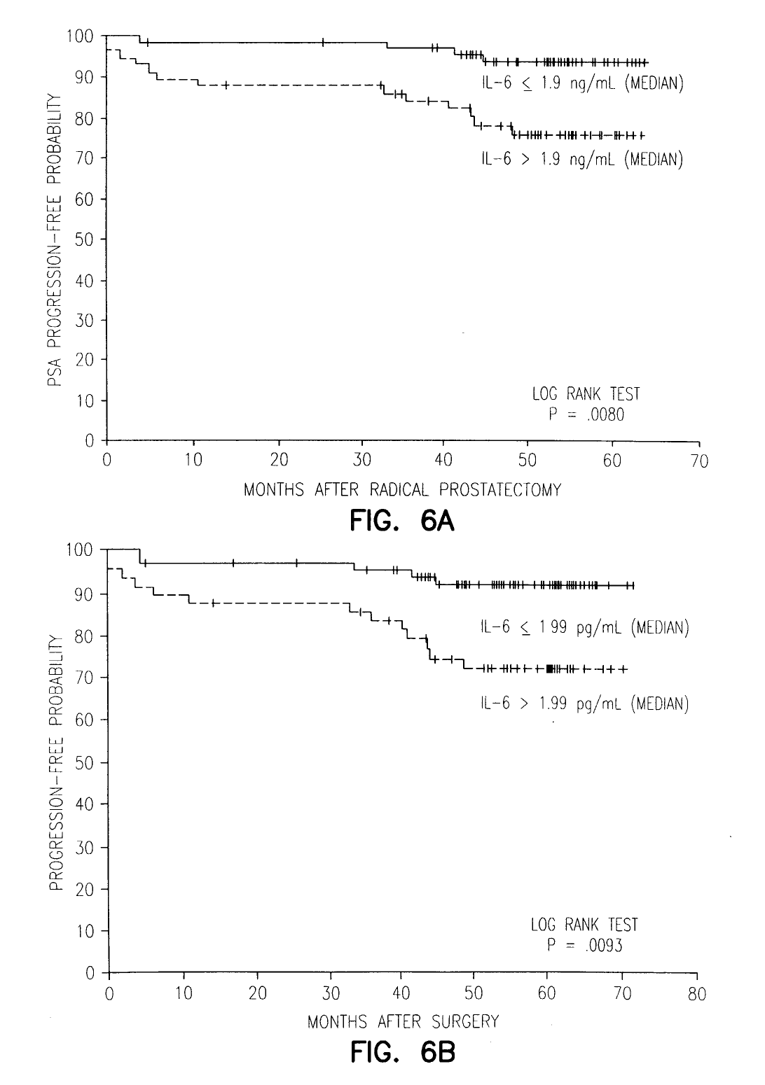

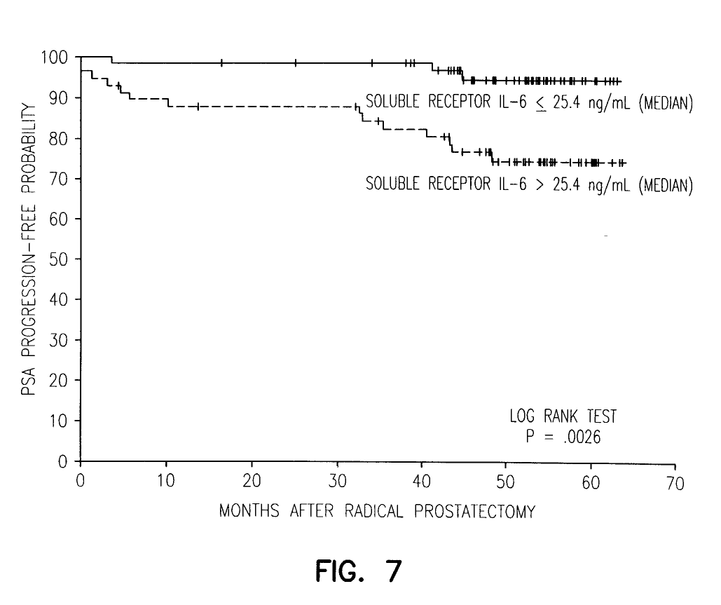

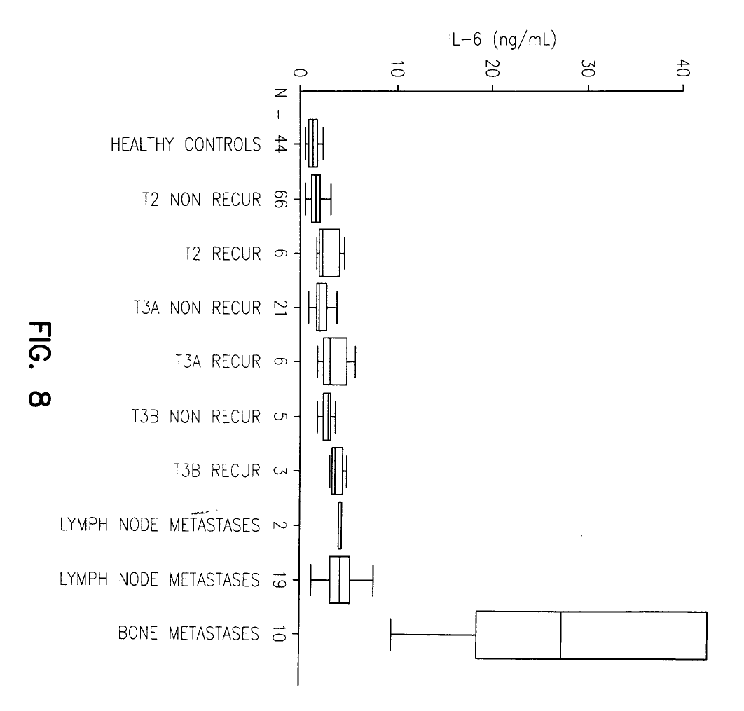

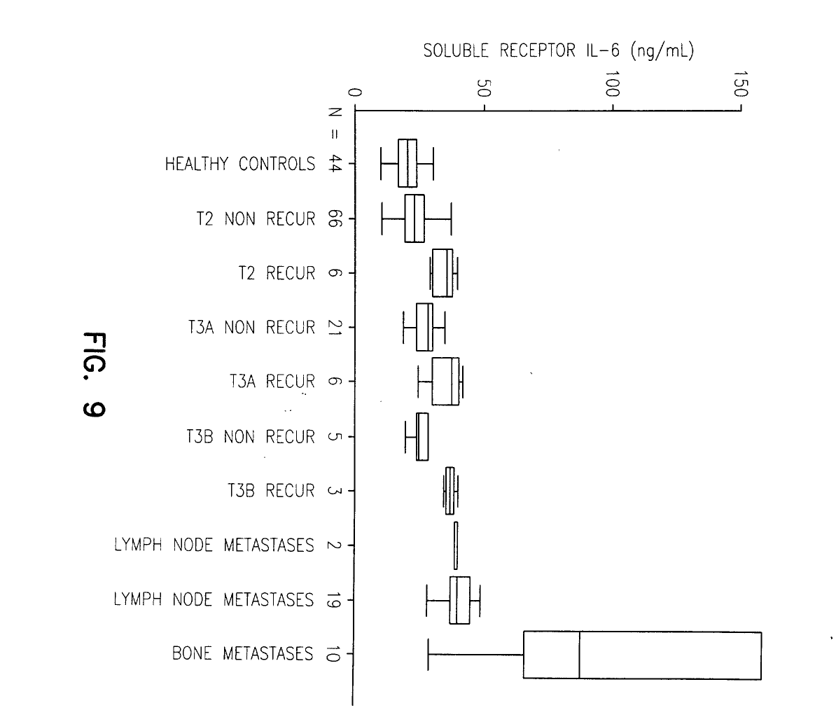

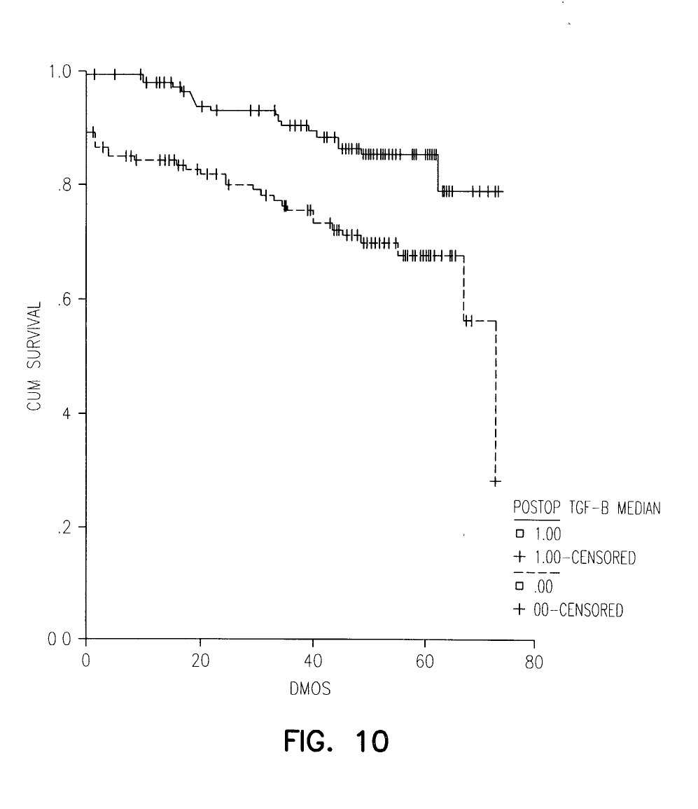

A similar analysis was conducted for IL-6 and IL-6sR (using R&D Systems Quantikine kits for IL-6 and IL-6sR, catalog numbers DR6050 and DR600, respectively) and it was found that the pre-operative plasma levels of IL-6 and IL-6sR were correlated with clinical and pathological parameters in the 120 patients who underwent radical prostatectomy (FIGS. 6-9 and Tables 11-12). Plasma IL-6 and IL-6sR levels in patients with bone metastases were significantly higher than those in healthy subjects, in prostatectomy patients, or in patients with lymph node metastases (P values<0.001). In a pre-operative model that included IL-6 or IL-6sR in addition to Partin nomogram variables, pre-operative plasma IL-6, IL-6sR, and biopsy Gleason score were independent predictors of organ-confined disease (P values<0.01) and PSA progression (P values<0.028). In an alternative model that included both IL-6 and IL-6sR, only pre-operative plasma IL-6sR remained an independent predictor of PSA progression (P=0.038). Thus, IL-6 and IL-6sR levels are elevated in men with prostate cancer metastatic to bone. In patients with clinically localized prostate cancer, the pre-operative plasma level of IL-6 and IL-6sR are associated with markers of more aggressive prostate cancer and are predictors of biochemical progression after surgery.

TABLE 11
Pre-operative Features

| | Univariate | | | Multivariate | | |
|---|---|---|---|---|---|---|
| | Hazard | | | Hazard | | |
| | ratio | P | 95% CI | ratio | P | 95% CI |
| Pre-operative PSA levels* | 5.772 | 0.067 | 0.887-37.547 | 4.197 | 0.131 | 0.652-27.017 |
| Pre-operative IL-6 levels | 2.291 | <0.001 | 1.678-3.128 | 1.226 | <0.001 | 1.114-1.3498 |
| Biopsy Gleason Sum† | 4.167 | 0.005 | 1.541-11.273 | 2.063 | 0.185 | 0.707-6.020 |
| Clinical Stage‡ | 1.850 | 0.226 | 0.684-5.002 | 1.085 | 0.977 | 0.347-2.798 |

*Pre-operative PSA levels were logarithmically transformed.
†Biopsy Gleason sum was categorized as grade 2 to 6 versus grade 7 to 10.
‡Clinical stage was categorized as T1 versus T2.

TABLE 12
Pre-operative Features

| | Univariate | | | Multivariate | | |
|---|---|---|---|---|---|---|
| | Hazard | | | Hazard | | |
| | ratio | P | 95% CI | ratio | P | 95% CI |
| Pre-operative PSA levels* | 5.772 | 0.067 | 0.887-37.547 | 7.083 | 0.044 | 1.051-47.726 |
| Pre-operative soluble receptor | 1.260 | <0.001 | 1.154-1.375 | 2.174 | <0.001 | 1.550-3.048 |
| IL-6 levels | | | | | | |
| Biopsy Gleason Sum† | 4.167 | 0.005 | 1.541-11.273 | 3.218 | 0.026 | 1.148-9.025 |
| Clinical Stage‡ | 1.850 | 0.226 | 0.684-5.002 | 1.135 | 0.814 | 0.396-3.254 |

*Preoperative PSA levels were logarithmically transformed.
†Biopsy Gleason sum was categorized as grade 2 to 6 versus grade 7 to 10.
‡Clinical stage was categorized as T1 versus T2.

### EXAMPLE 4

A cohort of 468 radical prostatectomy patients was employed to study marker interactions. Of these patients, 278 patients had samples available at 6 to 8 weeks after post-radical prostatectomy. The clinical stage of these patients was <T3a (47% cT1, 49% cT2, and 4% cT3a) and they had a median PSA of 8.2 ng/mL (range of 0.2 to 60 ng/mL). The median age for these patients was 63 years (range 40 to 81) and the median follow up for them was about 51 months. Fourteen percent (63/468) had PSA recurrence.

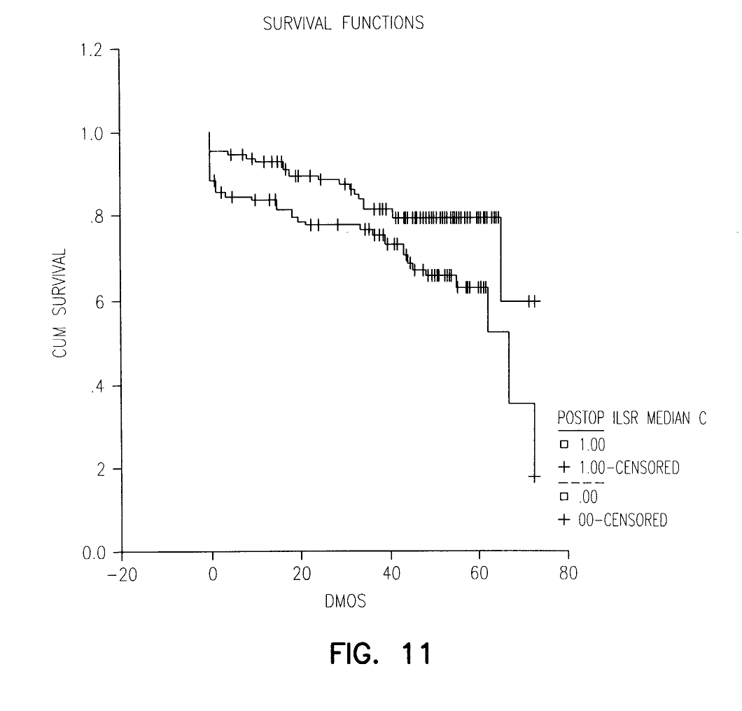

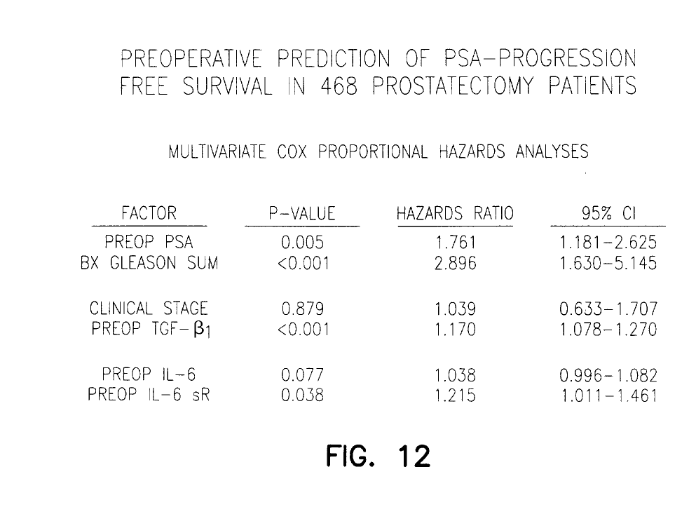

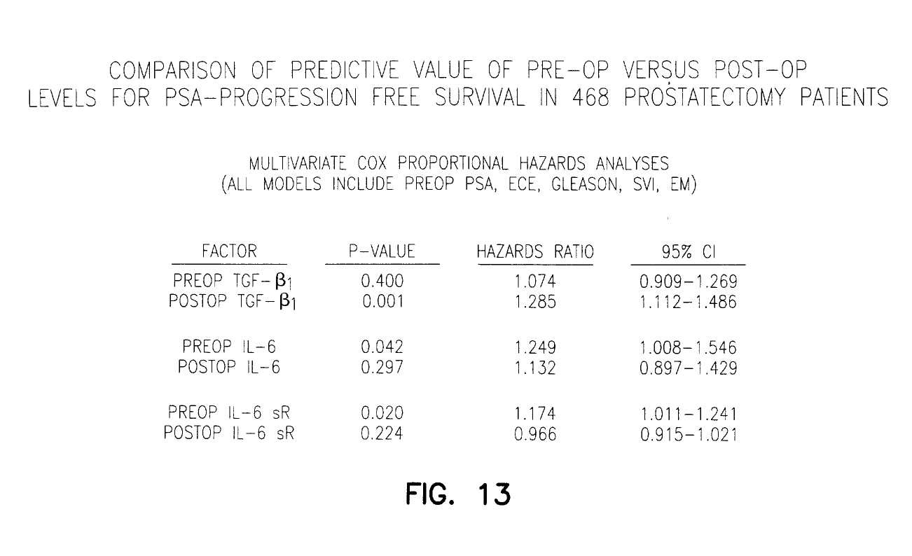

Post-operative plasma TGF-β₁ levels in these patients were found to be useful as a prognostic marker for prostate cancer progression (FIG. 10). Thus, serial measurements TGF-β₁ may be particularly useful to monitor the outcome of therapy, e.g., surgery, radiation, or hormonal therapy or brachytherapy, similary to serial measurements of PSA. Moreover, in a multivariate Cox proportional hazards model, post-therapy measurements of TGF-β₁ were found to be a stronger predictor of progression than pre-therapy measurements of TGF-β₁ (FIG. 13). Further, post-therapy levels of IL-6 and IL-6sR (FIG. 11 and FIG. 13), unlike TGF-β₁, were not significant predictors of progression.

TABLE 13
Multivariate Cox Regression
Variables in the Equation

| | | | | | | | 95% Cl for Exp(B) | |
|---|---|---|---|---|---|---|---|---|
| | B | SE | Wald | df | Sig. | Exp(B) | Lower | Upper |
| SVI | .078 | .357 | .047 | 1 | .828 | 1.081 | .536 | 2.178 |
| MARGINS | .614 | .321 | 3.660 | 1 | .056 | 1.849 | .985 | 3.469 |
| LNPSA | .435 | .227 | 3.679 | 1 | .055 | 1.545 | .991 | 2.409 |
| GGTOT_G | 1.479 | .468 | 9.981 | 1 | .002 | 4.388 | 1.753 | 10.982 |
| ECE_KAT | .129 | .277 | .216 | 1 | .642 | 1.137 | .661 | 1.957 |
| TGF_POST | .296 | .046 | 41.825 | 1 | .000 | 1.345 | 1.229 | 1.471 |

Kaplan-Meier
Survival Analysis for DMOS (follow-up time since surgery)
Factor P_TGF_M = .00

| | Survival Time | Standard Error | 95% Confidence Interval |
|---|---|---|---|
| Mean: | 65.36233 | 1.62277 | (62.18171, 68.54295) |
| (Limited to | | | |
| 72.567) | | | |

Survival Analysis for DMOS (follow-up time since surgery)
Factor P_TGF_M = 1.00

| | Survival Time | Standard Error | 95% Confidence Interval |
|---|---|---|---|
| Mean: | 54.53912 | 2.47776 | (49.68272, 59.39553) |
| (Limited to | | | |
| 72.467) | | | |
| Median: | 72.26667 | 4.13222 | (64.16752, 80.36582) |

Survival Analysis for DMOS (follow-up time since surgery)

| | | | Number | Number | Percent |
|---|---|---|---|---|---|
| | | Total | Events | Censored | Censored |
| P_TGF_M | .00 | 142 | 18 | 124 | 87.32 |
| P_TGF_M | 1.00 | 136 | 39 | 97 | 71.32 |
| Overall | | 278 | 57 | 221 | 79.50 |

Test Statistics for Equality of Survival Distributions for P_TGF_M

| | Statistic | df | Significance |
|---|---|---|---|
| Log Rank | 11.88 | 1 | .0006 |

TABLE 14
Multivariate Cox Regression
Variables in the Equation

| | | | | | | | 95% Cl for Exp(B) | |
|---|---|---|---|---|---|---|---|---|
| | B | SE | Wald | df | Sig. | Exp(B) | Lower | Upper |
| SVI | .503 | .364 | 1.910 | 1 | .167 | 1.653 | .810 | 3.372 |
| MARGINS | .288 | .312 | .852 | 1 | .356 | 1.334 | .724 | 2.457 |
| LNPSA | .416 | .231 | 3.237 | 1 | .072 | 1.516 | .963 | 2.386 |
| GGTOT_G | 1.086 | .450 | 5.813 | 1 | .016 | 2.961 | 1.225 | 7.157 |
| ECE_KAT | .402 | .272 | 2.189 | 1 | .139 | 1.495 | .878 | 2.547 |
| ILSR_POS | .219 | .104 | 4.410 | 1 | .036 | 1.245 | 1.015 | 1.528 |

Kaplan-Meier
Survival Analysis for DMOS (follow-up time since surgery)
Factor P_ILSR_M = .00

| | Survival Time | Standard Error | 95% Confidence Interval |
|---|---|---|---|
| Mean: | 59.79814 | 2.50355 | (54.89119, 64.70509) |
| (Limited to | | | |
| 72.233) | | | |

Survival Analysis for DMOS (follow-up time since surgery)
Factor P_ILSR_M = 1.00

| | Survival Time | Standard Error | 95% Confidence Interval |
|---|---|---|---|
| Mean: | 51.02185 | 2.87315 | (45.39047, 56.65323) |
| (Limited to | | | |
| 72.467) | | | |
| Median: | 66.63333 | 6.76281 | (53.37824, 79.88843) |

Survival Analysis for DMOS (follow-up time since surgery)

| | | | Number | Number | Percent |
|---|---|---|---|---|---|
| | | Total | Events | Censored | Censored |
| P_ILSR_M | .00 | 111 | 21 | 90 | 81.08 |
| P_ILSR_M | 1.00 | 110 | 37 | 73 | 66.36 |
| Overall | 221 | 58 | 163 | 73.76 | |

Test Statistics for Equality of Survival Distributions for P_ILSR_M

| | Statistic | df | Significance |
|---|---|---|---|
| Log Rank | 5.37 | 1 | 0.0204 |

Thus, from an extensive analysis of a large cohort of patients, it was determined that measuring the TGF-beta levels in prostate cancer patients just after therapy, e.g., just after the prostate is removed at radical prostatectomy, can be used in addition to measuring TGF-beta levels prior to surgery, or may be used alone, for predicting disease recurrence including aggressive disease recurrence.

References

Adler H L, McCurdy M A, Kattan M W, et al: Elevated levels of circulating interleukin-6 and transforming growth factor-betal in patients with metastatic prostatic carcinoma. J Urol 161: 182-7, 1999.

Catalona W J, and Smith D S: Cancer recurrence and survival rates after anatomic radical retropubic prostatectomy for prostate cancer: intermediate-term results. J Urol 160: 2428-34, 1998.

Coalo, J. Clin. Endocrinol. Metab., 83:775, 1998.

Danielpour D, et al.: Evidence for differential regulation of TGFβ1 and TGFβ2 expression in vivo by sandwich enzyme-linked immunosorbent assays. Annals New York Academy of Sciences, pp. 300-302, 1990.

Danielpour D, et al.: Immunodetection and quantitation of the two forms of transforming growth factor-beta (TGF-β1 and TGF-β2) secreted by cells in culture. Journal of Cellular Physiology 138: 79-86, 1989.

Danielpour D: Improved sandwich enzyme-linked immunosorbent assays for transforming growth factor β₁. Journal of immunological methods 158: 17-25, 1993.

Dasch J R, et al.: Capture immunoassays specific for TGF-β1 and TGF-β2: Use in pharmacokinetic studies. Annals New York Academy of Sciences, pp. 303-305, 1990.

Eastham J, and Scardino P: Radical prostatectomy for clinical stage T1 and T2 prostate cancer. In: N J Vogelzang, P T Scardino, W U Shipley, and D S Coffey (eds), Comprehensive textbook of genitourinary oncology, pp 722-738 Maryland: Wiliams and Wilkins, 2000.

Eastham J A, Truong L D, Rogers E, et al: Transforming growth factor-beta 1: comparative immunohistochemical localization in human primary and metastatic prostate cancer. Lab Invest 73: 628-35, 1995.

Eder I E, Stenzl A, Hobisch A, et al: Transforming growth factors-beta 1 and beta 2 in serum and urine from patients with bladder carcinoma [see comments]. J Urol 156: 953-7, 1996.

Engeler C E, Wasserman N F, and Zhang G: Pre-operative assessment of prostatic carcinoma by computerized tomography. Weaknesses and new perspectives. Urology 40: 346-50, 1992.

Epstein J I, Partin A W, Potter S R, et al: Adenocarcinoma of the prostate invading the seminal vesicle: prognostic stratification based on pathologic parameters. Urology 56: 283-8, 2000.

Epstein J I, Pound C R, Partin A W, et al: Disease progression following radical prostatectomy in men with Gleason score 7 tumor [see comments]. J Urol 160: 97-100; discussion 101, 1998.

Epstein J I, Partin A W, Sauvageot J, et al: Prediction of progression following radical prostatectomy. A multivariate analysis of 721 men with long-term follow-up. Am J Surg Pathol 20: 286-92, 1996.

Farkas A, Schneider D, Perrotti M, et al: National trends in the epidemiology of prostate cancer, 1973 to 1994: evidence for the effectiveness of prostate-specific antigen screening. Urology 52: 444-9, 1998.

Flanders K C: Transforming Growth Factor-β1: Histochemical localization with antibodies to different epitopes. The Journal of Cell Biology 108: 653-660, 1989.

Ivanovic V, Melman A, Davis-Joseph B, et al: Elevated plasma levels of TGF-beta 1 in patients with invasive prostate cancer [letter]. Nat Med 1: 282-4, 1995.

Jones E C: Resection margin status in radical retropubic prostatectomy specimens: relationship to type of operation, tumor size, tumor grade and local tumor extension. J Urol 144: 89-93, 1990.

Junker U, Knoefel B, Nuske K, et al: Transforming growth factor beta 1 is significantly elevated in plasma of patients suffering from renal cell carcinoma. Cytokine 8: 794-8, 1996.

Kakehi Y, Oka H, Mitsumori K, et al: Elevation of serum transforming growth factor beta 1 level in patients with metastatic prostate cancer. Urol Oncol 2: 131-135, 1996.

Kattan M W, Eastham J A, Stapleton A M, et al: A pre-operative nomogram for disease recurrence following radical prostatectomy for prostate cancer. J Natl Cancer Inst 90: 766-71, 1998.

Kong F, Jirtle R L, Huang D H, et al: Plasma transforming growth factor-beta 1 level before radiotherapy correlates with long term outcome of patients with lung carcinoma. Cancer 86: 1712-9, 1999.

Lucas C, et al.: The autocrine production of transforming growth factors-β₁ during lymphocyte activation. The Journal of Immunology 145: 1415-1422, 1990.

Maru N, Utsunomiya T, Bergamaschi F, et al: The influence of pathological parameters of positive surgical margins on prognosis in patients undergoing radical prostatectomy. J Urol 161: A 1280, 1999.

Merrill R M, Stephanson R A: Trends in mortality rates in patients with prostate cancer during the era of prostate specific antigen screening. J Urol., 163: 503-10, 2000.

Newcomer L M, Stanford J L, Blumenstein B A, et al: Temporal trends in rates of prostate cancer: declining incidence of advanced stage disease, 1974 to 1994. J Urol 158: 1427-30, 1997.

Nunn et al., J. Cell Physiol., 171:196, 1997.

Oesterling J E, Martin S K, Bergstralh E J, et al: The use of prostate-specific antigen in staging patients with newly diagnosed prostate cancer [see comments]. JAMA 269: 57-60, 1993.

Ohori M, Wheeler T M, Kattan M W, et al: Prognostic significance of positive surgical margins in radical prostatectomy specimens. J Urol 154: 1818-24, 1995.

Partin A W, Kattan M W, Subong E N, et al: Combination of prostate-specific antigen, clinical stage, and Gleason score to predict pathological stage of localized prostate cancer. A multi-institutional update [see comments] [published erratum appears in JAMA 278(2):118, 1997]. JAMA 277: 1445-51, 1997.

Partin A W, Pound C R, Clemens J Q, et al: Serum PSA after anatomic radical prostatectomy. The Johns Hopkins experience after 10 years. Urol Clin North Am 20: 713-25, 1993.

Perry K T, Anthony C T, Case T, et al: Transforming growth factor beta as a clinical biomarker for prostate cancer. Urology 49: 151-5, 1997.

Pound C R, Partin A W, Eisenberger M A, et al: Natural history of progression after PSA elevation following radical prostatectomy [see comments]. JAMA 281: 1591-7, 1999.

Shirai Y, Kawata S, Tamura S, et al: Plasma transforming growth factor-beta 1 in patients with hepatocellular carcinoma. Comparison with chronic liver diseases. Cancer 73: 2275-9, 1994.

Smith D S, Catalona W J, and Herschman J D: Longitudinal screening for prostate cancer with prostate-specific antigen [see comments]. JAMA 276: 1309-15, 1996.

Steiner M S, and Barrack E R: Transforming growth factor-beta 1 overproduction in prostate cancer: effects on growth in vivo and in vitro. Mol Endocrinol 6: 15-25, 1992.

Sutkowski et al., J Natl. Cancer Inst., 91: 1663, 1991.

Tefilli M V, Gheiler E L, Tiguert R, et al: Prognostic indicators in patients with seminal vesicle involvement following radical prostatectomy for clinically localized prostate cancer. J Urol 160: 802-6, 1998.

Thompson N L, et al.: Expression of transforming growth factor-β1 in specific cells and tissues of adult and neonatal mice. The Journal of Cell Biology 108: 661-669, 1989.

Thompson T C, Truong L D, Timme T L, et al: Transforming growth factor beta 1 as a biomarker for prostate cancer. J Cell Biochem Suppl 54-61, 1992.

Truong L D, Kadmon D, McCune B K, et al: Association of transforming growth factor-beta 1 with prostate cancer: an immunohistochemical study. Hum Pathol 24: 4-9, 1993.

Wakefield L M, Letterio J J, Chen T, et al: Transforming growth factor-betal circulates in normal human plasma and is unchanged in advanced metastatic breast cancer. Clin Cancer Res 1: 129-36, 1995.

Walsh P C, Partin A W, and Epstein J I: Cancer control and quality of life following anatomical radical retropubic prostatectomy: results at 10 years [see comments]. J Urol 152: 1831-6, 1994.

Wheeler T M, and Lebovitz R M: Fresh tissue harvest for research from prostatectomy specimens. Prostate 25: 274-9, 1994.

Wheeler T M, Dillioglugil 0, Kattan M W, et al: Clinical and pathological significance of the level and extent of capsular invasion in clinical stage T1-2 prostate cancer. Hum Pathol 29: 856-62, 1998.

Wheeler T M: Anatomic considerations in carcinoma of the prostate. Urol Clin North Am 16: 623-34, 1989.

Wolff J M, Fandel T H, Borchers H, et al: Serum concentrations of transforming growth factor-beta 1 in patients with benign and malignant prostatic diseases. Anticancer Res 19: 2657-9, 1999.

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A method to determine the risk of progression of a prostate cancer patient after therapy, comprising:
a) detecting or determining the amount or level of complex formation between a blood plasma sample obtained from a patient after therapy for prostate cancer and an agent that binds to TGF-β₁, IGFBP-2, or IGFBP-3, so as to form a complex; and
b) correlating the amount or level of complex formation with the risk of progression.

2. A method to determine the prognosis of a prostate cancer patient after therapy, comprising:
a) detecting or determining the amount or level of complex formation between a blood plasma sample obtained from a patient prior to or after therapy for clinically localized prostate cancer and an agent that binds to TGF-β₁, IGFBP-2, IGFBP-3, IL-6 or IL-6sR so as to form a complex; and
b) correlating the amount or level of complex formation with the risk of non-prostate confined disease.

3. The method of claim 1 or 2 wherein the sample is obtained at one to two months after therapy.

4. The method of claim 1 wherein the clinical stage of the patient is T3a, T3, T2c, T2b, T2a, T2, T1c, T1b, T1a or T1.

5. The method of claim 2 wherein the amount or level of complex formation is correlated to an increased risk of biochemical progression or to final pathological stage.

6. The method of claim 1 or 2 wherein the therapy is primary therapy.

7. The method of claim 1 or 2 wherein the agent is an antibody.

8. The method of claim 7 wherein the antibody is a polyclonal antibody.

9. The method of claim 7 wherein the antibody is a monoclonal antibody.

10. The method of claim 1 or 2 wherein the therapy is surgery, radical prostatectomy, radiation therapy or a radioactive seed implant.

11. The method of claim 1 or 2 wherein the cancer exhibits extracapsular extension orseminal vesicle involvement or wherein the patient does not have detectable metastases.

12. The method of claim 1 or 2 further comprising:
c) contacting a second blood sample obtained from the patient at a later point in time with the agent so as to form a complex; and
d) comparing complex formation in a) to complex formation in c) and determining whether the level or amount of complex formation in c) is different than the level or amount of complex formation in a).

13. The method of claim 1 or 2 further comprising detecting or determining the amount or level of a molecule other than TGF-β₁, IGFBP-2 or IGFBP-3 which is a marker for prostate cancer.

14. The method of claim 2 further comprising detecting or determining the amount or level of a molecule other than IL-6 or IL-6sR which is a marker for prostate cancer.

15. The method of claim 13 or 14 wherein the other marker is selected from the group consisting of PSA, UPA, UPAR, PAI-1, IGFBP-3, p53, p21, and E-cadherin.

16. The method of claim 13 or 14 wherein the other marker is a serum protein.

17. The method of claim 1 or 2 further comprising determining PSA levels or Gleason scores.

18. The method of claim 1 or 2 wherein the patient has not been subject to hormonal therapy.

19. The method of claim 1 or 2 wherein complex formation is detected or determined with an agent that specifically binds the complex.

20. The method of claim 1 wherein complex formation is detected or determined with a second agent that binds TGF-β₁, IGFBP-2, or IGFBP-3.

21. The method of claim 2 wherein complex formation is detected or determined with a second agent that binds TGF-β₁, IGFBP-2, IL-6, IL-6sR or IGFBP-3.

22. The method of claim 20 or 21 wherein the agent is an antibody.

23. The method of claim 1 or 2 wherein the agent is detectably labeled or binds to a detectable label.

24. The method of claim 19 wherein the agent that binds the complex is detectably labeled or binds to a detectable label.

25. The method of claim 7 wherein the antibody is detectably labeled or binds to a detectable label.

26. The method of claim 1 or 2 wherein the correlating of the amount or level of complex formation to the risk is conducted by a computer.

27. The method of claim 1 or 2 wherein the blood plasma sample is a platelet poor plasma sample.

28. An apparatus, comprising:
a data input means, for input of test information comprising the level or amount of at least one protein in a sample obtained from a mammal, wherein the protein is selected from the group consisting of TGF-β₁, IGFBP-2, and IGFBP-3;
a processor, executing a software for analysis of the level or amount of the at least one protein in the sample;
wherein the software analyzes the level or amount of the at least one protein in the sample and provides the risk of disease progression in the mammal.

29. An apparatus, comprising:
a data input means, for input of test information comprising the level or amount of at least one protein in a sample obtained from a mammal, wherein the protein is selected from the group consisting of TGF-β₁, IGFBP-2, IL-6, IL-6sR and IGFBP-3;
a processor, executing a software for analysis of the level or amount of the at least one protein in the sample;
wherein the software analyzes the level or amount of the at least one protein in the sample and provides the risk of non-prostate confined disease in the mammal.

30. The apparatus of claim 28 or 29 wherein the amount or level is input manually using the data input means.

31. The apparatus of claim 28 or 29 wherein the software constructs a database of the test information.

32. The apparatus of claim 28 or 29 wherein the information farther comprises the amount or level of PSA, UPA, UPAR, PAI-1, p53, p21, and E-cadherin.

33. The apparatus of claim 28 or 29 wherein the information further comprises a Gleason score.

34. A method to determine the prognosis of a prostate cancer patient after therapy, comprising:
a) inputting test information to a data input means, wherein the information comprises the level or amount of at least one protein in a sample obtained from a patient, and wherein the protein is selected from the group consisting of TGF-β₁, IGFBP-2 and IGFBP-3;
b) executing a software for analysis of the test information; and
c) analyzing the test information so as to provide the risk of disease progression in the patient.

35. A method to determine the prognosis of a prostate cancer patient after therapy, comprising:
a) inputting test information to a data input means, wherein the information comprises the level or amount of at least one protein in a sample obtained from a patient, and wherein the protein is selected from the group consisting of TGF-β₁, IGFBP-2, IL-6, IL-6sR and IGFBP-3;
b) executing a software for analysis of the test information; and
c) analyzing the test information so as to provide the risk of non-prostate confined disease in the patient.
